(19) United States
(12) Patent Application Publication
(10) Pub. No.: US 2025/0276992 A1
(43) Pub. Date: Sep. 4, 2025

(54) SYNTHESIS METHODS OF ACYL CHAIN OF QS-21 AND DERIVATIVE THEREOF

(71) Applicant: OBI Pharma, Inc., Taipei City (TW)

(72) Inventors: Ming-Tain LAI, Taipei City (TW); Jhao-Long LIN, Taipei City (TW); Wei-Han LEE, Taipei City (TW); Bing-Cheng CHOU, Taipei City (TW)

(73) Assignee: OBI Pharma, Inc., Taipei City (TW)

(21) Appl. No.: 18/862,675

(22) PCT Filed: May 5, 2023
(86) PCT No.: PCT/US2023/021095

§ 371 Date: Nov. 4, 2024

Related U.S. Application Data
(60) Provisional application No. 63/338,707, filed on May 5, 2022.

(51) Int. Cl. C07H 15/04; C07C 45/56; C07C 67/08; C07D 263/24; C07D 339/08; C07H 1/00; C07H 7/06

(52) U.S. Cl. CPC C07H 15/04; C07C 45/567; C07C 67/08; C07D 263/24; C07D 339/08; C07H 1/00; C07H 7/06

(57) ABSTRACT

The present disclosure provides synthesis methods used in the preparation of an acyl chain of QS-21 and its derivative.

### FIELD OF INVENTION

The present disclosure relates to a preparation method of QS-21, and in particular, synthesis methods of an acyl chain used in the preparation of QS-21.

### BACKGROUND OF THE INVENTION

Saponin is a type of compound extracted from Quillaja Saponaria Molina bark. Previous study showed several saponins, designated as QS-7, QS-17, QS-18, and QS-21, are able to dramatically boost antibody levels, which implied their pharmaceutical application as adjuvants. Among them, QS-21 is the prevalent adjuvant used in vaccine development.

QS-21 is an FDA approved saponin adjuvant that exhibits profound immunomodulating effect. Nature source of QS-21 is purified from the plant extract of the soap bark tree (Quillaja Saponaria Molina). Based on scarcity of Quillaja Saponaria trees and low-yield in the purification process, a scalable synthetic approach for manufacturing QS-21 can reduce dependency on nature source and secure constant supply of QS-21.

### SUMMARY OF THE INVENTION

The present invention provides synthesis methods of an acyl chain and a linear tetrasaccharide capable of being used in the preparation of QS-21.

In an aspect of the present invention, a method of preparing a compound of Formula I includes:

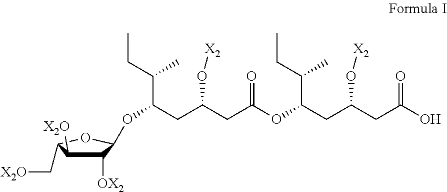

(a) performing hydroxylation and esterification of L-isoleucine to obtain compound AC-2;

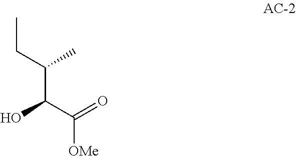

(b) treating compound AC-2 by intramolecular epoxidation and addition of 1,3-dithiane with an inversion process to obtain compound AC-6;

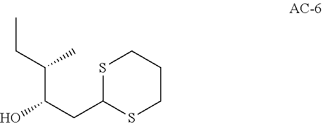

(c) installing a first alcohol protecting group to compound AC-6 and unmasking thio-protection to obtain Formula I-1

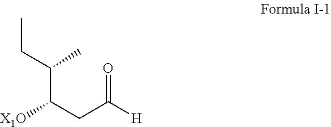

(d) performing an aldol reaction of Formula I-1 with compound (S)-Aux. by SmI₂ to obtain Formula I-2;

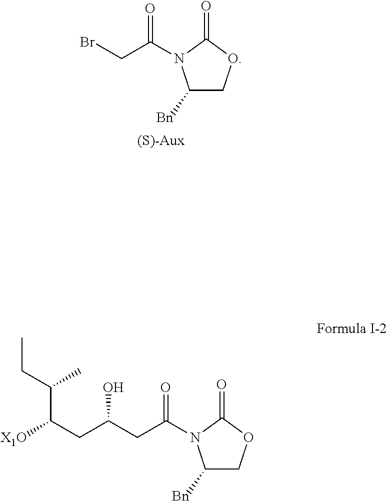

(e) installing a second alcohol protecting group to Formula I-2 to obtain Formula I-3; and

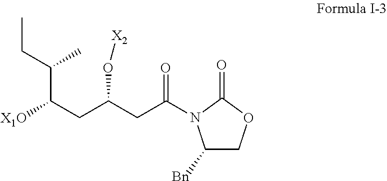

(f) removing the first alcohol protecting group from Formula I-3 via hydrogenolysis to obtain Formula I-4,

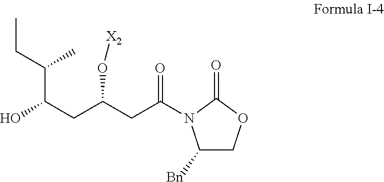

wherein X₁ is the first alcohol protecting group, and X₂ is the second alcohol protecting group.

In another aspect of the present invention, a method of preparing a compound of Formula IA is provided, wherein apart from replacing (S)-Aux. by (R)-Aux., the remaining steps are the same as the method of preparing a compound of Formula I described above.

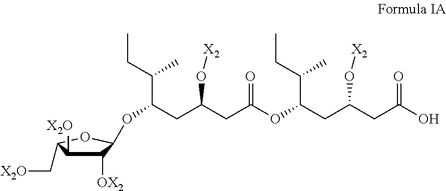

In another aspect of the present invention, a method of preparing a compound of Formula IB is provided, wherein apart from replacing hydroxylation by acetylation and without the inversion process, the remaining steps are the same as the method of preparing a compound of Formula I described above.

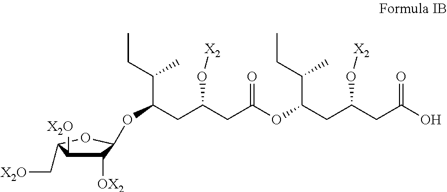

In another aspect of the present invention, a method of preparing a compound of Formula IC is provided, wherein apart from replacing hydroxylation by acetylation, replacing (S)-Aux. by (R)-Aux. and without the inversion process, the remaining steps are the same as the method of preparing a compound of Formula I described above.

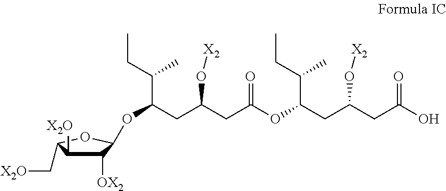

In another aspect of the present invention, a method of preparing a compound of Formula ID is provided, wherein apart from replacing L-isoleucine by D-isoleucine, replacing hydroxylation by acetylation and without the inversion process, the remaining steps are the same as the method of preparing a compound of Formula I described above.

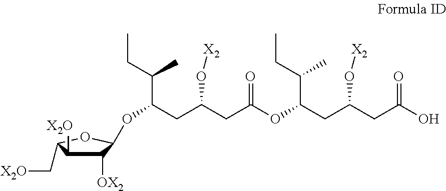

In another aspect of the present invention, a method of preparing a compound of Formula IE is provided, wherein apart from replacing L-isoleucine by D-isoleucine, replacing hydroxylation by acetylation, replacing (S)-Aux by (R)-Aux. and without the inversion process, the remaining steps are the same as the method of preparing a compound of Formula I described above.

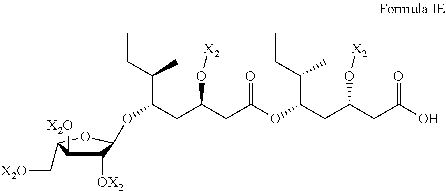

In another aspect of the present invention, a method of preparing a compound of Formula IF is provided, wherein apart from replacing L-isoleucine by D-isoleucine, the remaining steps are the same as the method of preparing a compound of Formula I described above.

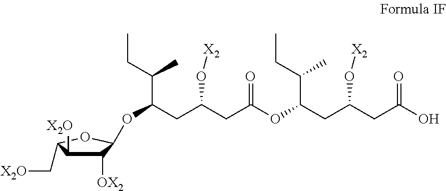

In another aspect of the present invention, a method of preparing a compound of Formula IG is provided, wherein apart from replacing L-isoleucine by D-isoleucine and replacing (S)-Aux. by (R)-Aux., the remaining steps are the same as the method of preparing a compound of Formula I described above.

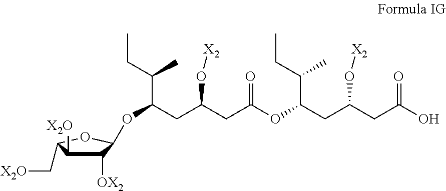

Preferably, X₁, X₂, X₃ and X₄ each are individually and independently selected from a group consisting of methyl, benzyl (Bn), p-methoxybenzyl (PMB), methoxymethyl (MOM), methoxyethoxymethyl (MEM), trimethylsilyl (TMS), triethylsilyl (TES), triisopropylsilyl (TIPS), tert-butyldimethylsilyl (TBDMS), tert-butyldiphenylsilyl (TBS/TBDPS) and tetrahydropyranyl (THP).

Given that the ratio of composition shifts with climate, the present invention provides cost-efficient synthetic methods as so to give a reliable source of QS-21 with consistent composition and purity.

### BRIEF DESCRIPTION OF THE DRAWINGS

FIG. 1 illustrates QS-21 structure.

FIG. 2 illustrates a synthesis process of acyl chain from L-isoleucine.

FIG. 3 illustrates the X-ray structure of compound AC-9.

FIG. 4A-4D illustrate the synthesis processes of four acyl chain derivatives having from L-isoleucine, wherein the four acyl chain derivatives have stereochemistry forms (3′S, 5′S, 6′S, 3S, 5S, 6S) (FIG. 4A); (3′S, 5′S, 6′S, 3R-OTES, 5S, 6S) (FIG. 4B); (3′S, 5′S, 6′S, 3S, 5R, 6S) (FIG. 4C); (3′S, 5′S, 6′S, 3R, 5R, 6S) (FIG. 4D) of compound AC-15, respectively.

FIG. 5A-5D illustrate the synthesis processes of four acyl chain derivatives from D-isoleucine, wherein the four acyl chain derivatives have stereochemistry forms (3′S, 5′S, 6′S, 3S, 5S, 6R) (FIG. 5A); (3′S, 5′S, 6′S, 3R, 5S, 6R) (FIG. 5B); (3′S, 5′S, 6′S, 3S, 5R, 6R) (FIG. 5C); (3′S, 5′S, 6′S, 3R, 5R, 6R) (FIG. 5D) of compound AC-15, respectively.

### DETAILED DESCRIPTION

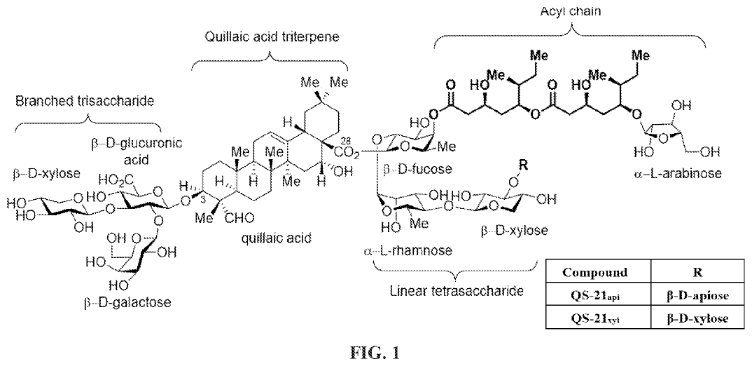

OBI-821 is a saponin-based adjuvant developed by OBI Pharma. Inc. and it is derived from the bark of the Quillaja saponaria (QS) Molina tree. FIG. 1 indicated the chemical structure of QS-21. It consists of a quillaic acid triterpene substituted with a branched trisaccharide and a linear tetrasaccharide, and the linear tetrasaccharide is connected to an acyl chain via a hydrolytically labile ester. OBI-821 is structurally similar to QS-21 based on the comparison of physicochemical data. Both OBI-821 and QS-21 exist as mixtures of isomers. OBI-821 is served as an immunological adjuvant that could potentiate the humoral antibody response to OBI-822 and OBI-833 vaccines. The detail of OBI-821 adjuvant is as disclosed in PCT publication number WO2019/191317.

The spotlight of L-isoleucine approach is cost-effective and high optical purity of the repeating unit in acyl chain. To keep well control of each chiral center, intramolecular epoxide formation is used to convert stereochemistry but retain optical purity from L-isoleucine. High chiral selectivity (>95%) of aldol reaction was employed to generate the carbon structure of repeating unit in acyl chain. The new approach described here significantly reduced undesired chiral reaction product, which greatly simplifies the purification process and facilitates the synthesis of the acyl chain of QS-21.

In an embodiment, a method of preparing a compound of Formula I includes:

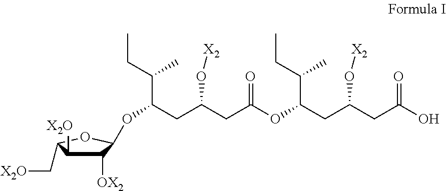

(a) performing hydroxylation and esterification of L-isoleucine to obtain compound AC-2:

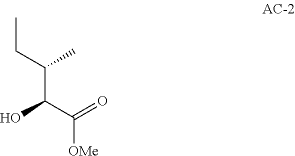

(b) treating compound AC-2 by intramolecular epoxidation and addition of 1,3-dithiane with an inversion process to obtain compound AC-6;

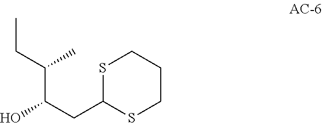

(c) installing a first alcohol protecting group to compound AC-6 and unmasking thio-protection to obtain Formula I-1

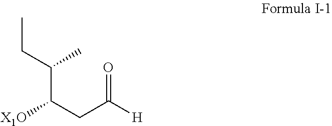

(d) performing an aldol reaction of Formula I-1 with compound (S)-Aux. by SmI₂ to obtain Formula I-2;

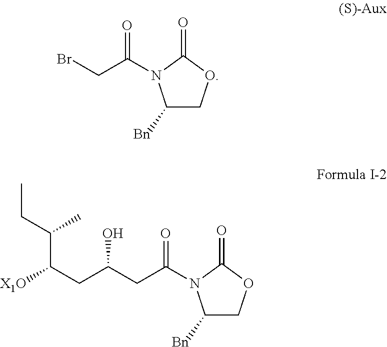

(e) installing a second alcohol protecting group to Formula I-2 to obtain Formula I-3;

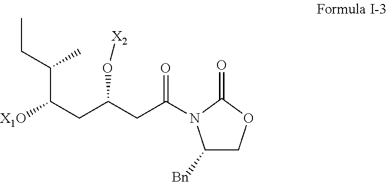

(f) removing the first alcohol protecting group from Formula I-3 via hydrogenolysis to obtain Formula I-4,

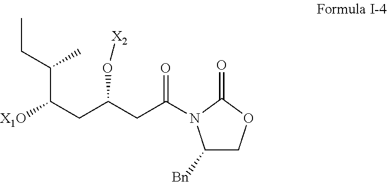

(g) performing glycosylation of Formula I-4 with Formula I-a to obtain Formula I-5

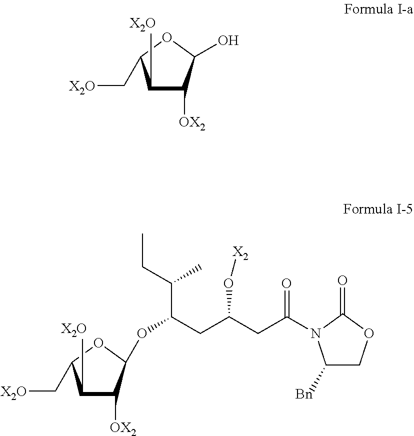

(h) treating Formula I-5 with lithium hydroxide and hydrogen peroxide to obtain Formula I-6;

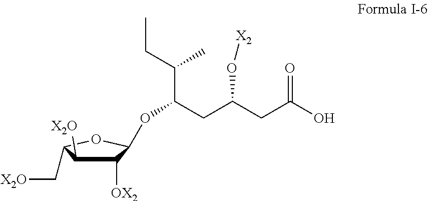

(i) coupling Formula I-6 with Formula I-b to obtain Formula I-7; and

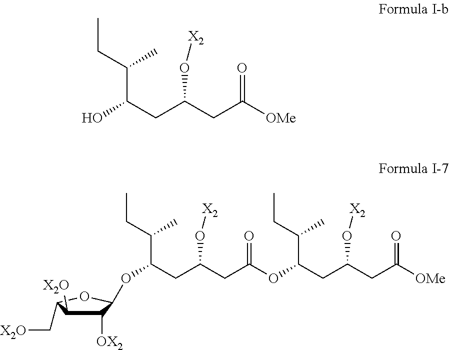

(j) performing hydrolysis of Formula I-7 to obtain Formula I,
wherein X₁ is the first alcohol protecting group, and X₂ is the second alcohol protecting group.

The term “alcohol protective group” indicates a functional group is temporarily attached to decrease reactivity so that the protected hydroxyl group does not react under synthetic conditions to which the molecule is subjected in one or more subsequent steps.

In one embodiment, each X₁ is independently selected from a group consisting of methyl, benzyl (Bn), p-methoxybenzyl (PMB), methoxymethyl (MOM), methoxyethoxymethyl (MEM), trimethylsilyl (TMS), triethylsilyl (TES), triisopropylsilyl (TIPS), tert-butyldimethylsilyl (TBDMS), tert-butyldiphenylsilyl (TBS/TBDPS) and tetrahydropyranyl (THP).

In one embodiment, each X₂ is independently selected from a group consisting of methyl, benzyl (Bn), p-methoxybenzyl (PMB), methoxymethyl (MOM), methoxyethoxymethyl (MEM), trimethylsilyl (TMS), triethylsilyl (TES), triisopropylsilyl (TIPS), tert-butyldimethylsilyl (TBDMS), tert-butyldiphenylsilyl (TBS/TBDPS) and tetrahydropyranyl (THP).

In one embodiment, each X₁ is benzyl (Bn) and each X₂ is TBS.

In one embodiment, Formula I-a is prepared by installing the second alcohol protecting groups to L-arabinose, and then selectively removing one of the second alcohol protecting groups.

In one embodiment, Formula I-b is prepared by treating Formula I-3 with dimethyl carbonate and sodium methoxide, and then treating with Pd/C and hydrogen.

In another embodiment, a method of preparing a compound of Formula IA is provided, wherein apart from replacing (S)-Aux. by (R)-Aux., the remaining steps are the same as the method of preparing a compound of Formula I described above. In one embodiment, each X₂ is TBS.

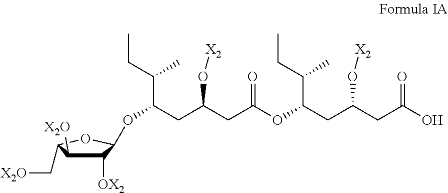

In another embodiment, a method of preparing a compound of Formula IB is provided, wherein apart from replacing hydroxylation by acetylation and without the inversion process, the remaining steps are the same as the method of preparing a compound of Formula I described above. In one embodiment, each X₂ is TBS; or one of X₂ is TES, and the remaining X₂ is TBS.

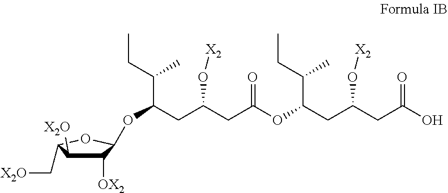

In another embodiment, a method of preparing a compound of Formula IC is provided, wherein apart from replacing hydroxylation by acetylation, replacing (S)-Aux. by (R)-Aux. and without the inversion process, the remaining steps are the same as the method of preparing a compound of Formula I described above. In one embodiment, each X₂ is TBS.

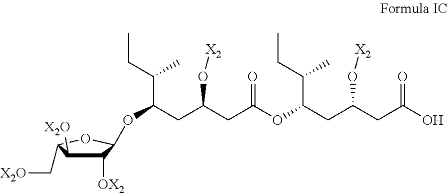

In another embodiment, a method of preparing a compound of Formula ID is provided, wherein apart from replacing L-isoleucine by D-isoleucine, replacing hydroxylation by acetylation and without the inversion process, the remaining steps are the same as the method of preparing a compound of Formula I described above. In one embodiment, each X₂ is TBS.

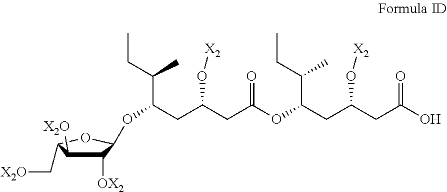

In another embodiment, a method of preparing a compound of Formula IE is provided, wherein apart from replacing L-isoleucine by D-isoleucine, replacing hydroxylation by acetylation, replacing (S)-Aux. by (R)-Aux. and without the inversion process, the remaining steps are the same as the method of preparing a compound of Formula I described above. In one embodiment, each X₂ is TBS.

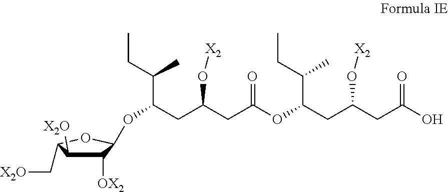

In another embodiment, a method of preparing a compound of Formula IF is provided, wherein apart from replacing L-isoleucine by D-isoleucine, the remaining steps are the same as the method of preparing a compound of Formula I described above. In one embodiment, each X₂ is TBS.

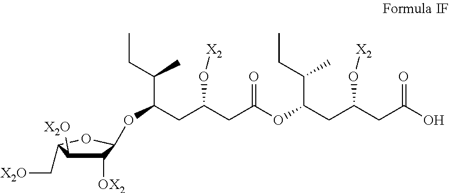

In another embodiment, a method of preparing a compound of Formula IG is provided, wherein apart from replacing L-isoleucine by D-isoleucine and replacing (S)-Aux. by (R)-Aux., the remaining steps are the same as the method of preparing a compound of Formula I described above. In one embodiment, each X₂ is TBS.

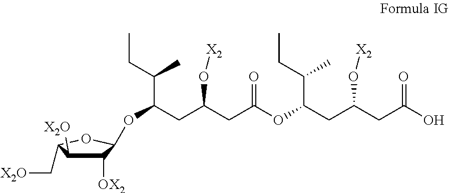

### EXAMPLES

Various features and embodiments of the disclosure are illustrated in the following representative examples, which are intended to be illustrative, and not limiting. Those skilled in the an will readily appreciate that the specific examples are only illustrative of the invention as described more fully in the claims which follow thereafter. Every embodiment and feature described in the present application should be understood to be interchangeable and combinable with emery embodiment contained within.

### Example 1: Synthesis of Acyl Chain

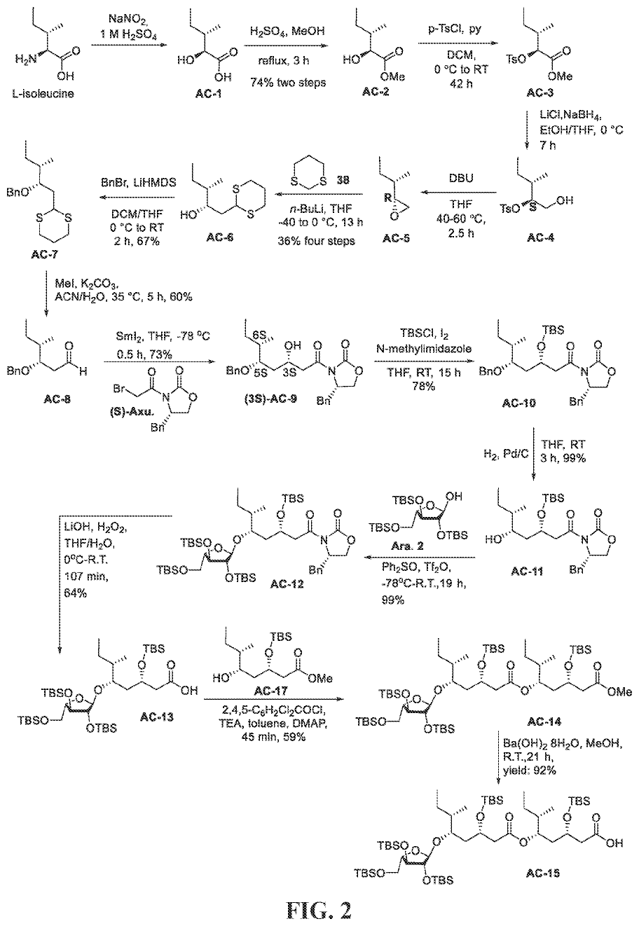

FIG. 2 indicated a synthesis method of an acyl chain. L-isoleucine is used as starting material to facilitate the synthesis process. Started from the commercial L-isoleucine, the key compound AC-11 was made with excellent stereoselective First. α-hydroxylation and esterification of L-isoleucine were performed to give methyl ester compound AC-2. Inversion of α-hydroxyl group and one carbon elongation were achieved via four sequential steps of intramolecular epoxidation and addition of 1,3-dithiane to give compound AC-6. Benzylation and unmasking thio-protection gave aldehyde compound AC-8. Aldol reaction of compound AC-8 and (S)-Aux by SmI₂ achieved excellent stereoselective of compound AC-9. X-ray crystallography analysis of compound AC-9 confirmed the absolute configuration of compound AC-9, suggesting that the present synthetic route is feasible. Followed TBS protection of hydroxyl group of compound AC-9 and sequential subjected of hydrogenolysis to remove Bn group, the key building block compound AC-Il was successfully obtained. Glycosylation with arabinose compound Ara. 2 was performed to have the desired α-form compound AC-12. After treatment of lithium hydroxide and hydrogen peroxide, the chiral auxiliary was removed to afford compound AC-13. Ester bond formation with compound AC-17 was performed to have acyl chain skeleton compound AC-14. Final step of acyl chain synthesis is hydrolysis of methyl ester. The overall yield of acyl chain synthesis is 2.1% with said steps from L-isoleucine.

### 1-1. Intermediate Compound AC-16

Compound AC-10 (0.20 g, 0.36 mmol, 1.0 equiv.) was diluted by dried MeOH (13.3 mL, 67 V). Dimethyl carbonate (DMC)(0.152 mL, 1.81 mmol, 5.0 equiv.) and NaOMe (0.0976 g, 1.81 mmol, 5.0 equiv.) were added to reaction and stirred for 110 min at room temperature. The reaction was quenched by 10 mL NH₄Cl. Methanol in reaction was removed by concentration at 35° C. After the concentrated residue extracted with DCM and dried over MgSO₄, the crude compound AC-16 (0.222 g) was in hand. The crude was purified with 25 g normal phase silica gel column eluted with 1%-5% EtOAc/hexane to get compound AC-16 (0.0851 g, 58%) as colorless oil. Rr 0.32 (EtOAc/Hexane 5/95); TLC stain: PMA; ¹H NMR (600 MHz, CDCl₃) δ 7.35-7.32 (m, 4H), 7.27-7.25 (m, 1H), 4.56 (d, J=11.5 Hz, 1H), 4.45 (d, J=11.5 Hz, 1H); ¹³C NMR (151 MHz, CDCl₃) δ 172.1, 139.3, 128.4, 127.5, 127.4, 80.5, 77.2, 71.0, 67.9, 51.6, 43.7, 39.0, 37.1, 29.9, 26.0, 24.2, 18.1, 15.1, 12.4, −4.3 ppm

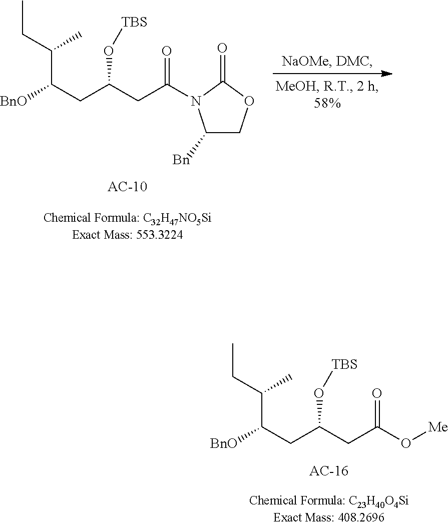

### 1-2. Intermediate Compound AC-17

Compound AC-16 (0.079 g, 0.193 mmol) was diluted by THF (1.6 mL, 20 V). Palladium on carbon (Pd/C) (10%, 0.158 g, 2 W) was added to reaction. The reaction flask was degassed and filled with hydrogen for 10 times. Hydrogenation was carried out with strong stirring for 3 hours at room temperature and balloon pressure. The reaction mixture was filtered through Celite®. The filtrate was concentrated at 35° C. to afford crude compound AC-17 (0.0702 g). The crude was purified by 25 g normal phase silica gel column which eluted by 5%-20% EtOAc/hexane to get compound AC-17 (0.0607 g, 99%) as colorless oil, R_{f} 0.16 (EtOAc/Hexane 1/9); TLC stain: PMA; ¹H NMR (600 MHz, CD₃OD) δ 4.36-4.32 (m, 1H), 3.68-3.66 (m, 4H), 2.54 (dd, J=14.6, 5.4 Hz, 1H), 2.49 (dd, J=14.6, 6.9 Hz, 1H), 1.62-1.58 (m, 1H), 1.56-1.46 (m, 2H), 1.35-1.30 (m, 1H), 1.18-1.11 (m, 1H), 0.91 (t, J=7.4 Hz, 3H), 0.88-0.86 (m, 12H), 0.11 (s, 3H), 0.07 (s, 3H); ¹³C NMR (151 MHz, CD₃OD) δ 173.5, 71.7, 68.7, 52.0, 44.2, 42.9, 42.1, 26.5, 26.3, 18.7, 14.2, 12.2 ppm.

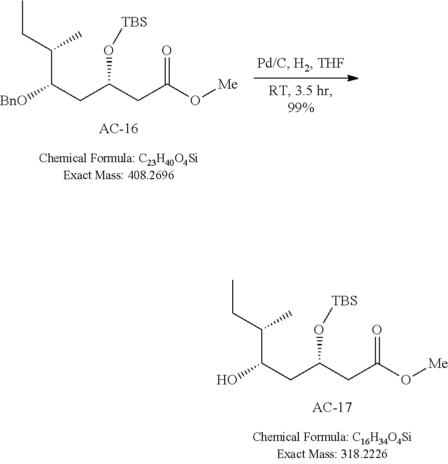

### 1-3. Intermediate Compound Ara. 1

L-Arabinose (5.0 g, 33.3 mmol) was dissolved in DMF (185 mL, 37 V) to which was added sequentially imidazole (12.59 g, 185 mmol, 5.56 equiv.) and tert-butyldimethylsilyl chloride (TBDMSCl) (26.8 g, 178 mmol, 5.34 equiv.). The mixture was then stirred at room temperature for 10 min before being heated at 70° C. for 2 hours. After it was cooled to 0° C. to 4° C. gently and stirred in the ice bath for 4 hours. The resultant white solids were filtered and recrystallized from 21.5 mL hot solution (about 46° C.) of chloroform and 5% ammonium hydroxide in methanol (CHCl₃: 5% NH₄OH/MeOH=1:4). The hot solution was slowly cooled to 0° C. in a period of 75 min. The product was then filtered off and dried in vacuo to yield product (15.8 g, 53%). ¹H NMR indicate product mix with TBDMSCl. Compound Ara. 1: R_{f}0.91 (DCM/Hexane 1/1); TLC stain: p-anisaldehyde. ¹H NMR (400 MHz, CDCl₃) δ 5.10 (s, 1H), 3.98 (dd, J=9.9 Hz, J=55 Hz, 1H), 3.88-3.91 (m, 2H), 3.64 (dd, J=5.6 Hz, J=1.8 Hz, 2H), 0.84-0.89 (m, 36H), 0.03-0.07 (m, 24H) ppm.

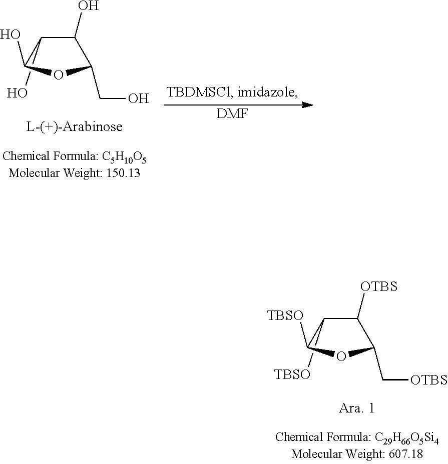

### 1-4. Intermediate Compound Ara. 2

At −20° C., trifluoroacetic acid (TFA) (4.7 mL, 9.4V) was added to a solution of starting material, compound Ara. 1 (0.50 g, 0.82 mmol) in chloroform (18.75 mL, 37.5V) in a period of 6 min. The mixture was stirred for 1 min before being poured into a stirred solution of ammonium hydroxide (NH₄OH)(9.4 mL, 18.75 V) in methanol (MeOH) (63 mL, 125.0 V) at −20° C. The mixture was warmed to room temperature and was added with dichloromethane (DCM) (100 mL, 250 V) and water (100 mL, 250 V). The organic layers were combined, dried over MgSO₄ and concentrated in vacuo to afford 0.451 g crude. Purification was carried out by silica gel column chromatography (20 g) by gradient elution (50 mL DCM/Hexane (25:75), 100 mL DCM/Hexane (30:70), 200 mL dichloromethane/Hexane (35:65), 200 mL DCM/Hexane (1:1) and 100 mL EA/Hexane (5:95)) to give compound Ara. 2 (0.328 g, 81%). Compound Ara. 2: R_{f} 0.14 (DCM/Hexane 30/70); TLC stain: p-anisaldehyde. ¹H NMR (400 MHz, CDCl₃) δ 5.34-5.04 (m, 1H), 4.19-3.54 (m, 6H), 0.91-0.86 (m, 27H), 0.12-0.02 (m, 18H) ppm

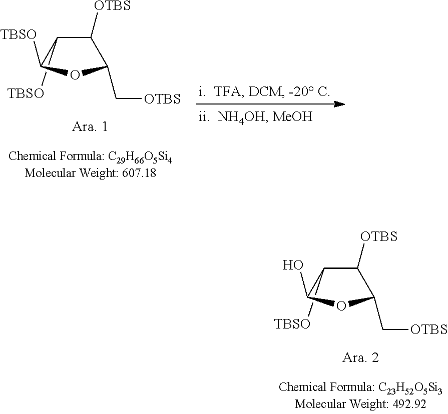

### 1-5. Intermediate Compound AC-1

A solution of NaNO₂ (42 g, 611 mmol) in H₂O (200 mL) was added dropwise over 4 hours to a cooled (ice bath) solution of L-isoleucine (10 g, 76.3 mmol) in H₂SO₄ (1.0 M, 200 mL) with stirring. The reaction temperature was kept below 5° C., and after completion of addition the mixture was stirred for another 8-10 h at less than 5° C. followed by stirred for four days at room temperature. The mixture was extracted with EA (4×125 mL). The combined organic phase was washed, dried over MgSO₄, filtered and evaporated to afford compound AC-1 (9.26 g, crude yield: 92%). Compound AC-1. R_{f} 0.54 (CHCl₃/MeOH/AcOH 95/20/3); TLC stain: PMA; LRMS (ESI-MS) calcd. for C₆H₁₁O₃ [M−H]⁻ 131, found 131.

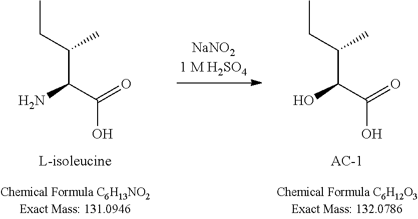

### 1-6. Intermediate Compound AC-2

Acid compound AC-1 was esterified by refluxing a solution of (S)-2-hydroxy-3-methylbutanoic acid (9.26 g, 70.10 mmol) in MeOH (93 mL) with a catalytic amount of concentrated H₂SO₄ (1.8 mL) for 3 h. The reaction was concentrated in vacuo, and diluted with dichloromethane (DCM) (37 mL), brine (9.4 mL) and H₂O (9.4 mL). The organic phase was dried (MgSO₄), filtered, and concentrated at 30° C. to 35° C., 36-26 cmHg. The crude oil (14.1 g) was then distilled in vacuo (external temp. 42° C. to 66° C., 2 mmHg) to afford methyl (S)-2-hydroxy-3-methylbutanoate compound AC-2 (8.3 g, 74% yield over 2 steps) as a colorless liquid. Compound AC-2: R_{f} 0.66 (EtOAc/Hexane 40/60); TLC stain: PMA; ¹H NMR (400 MHz, CDCl₃) δ 0.89 (t, J=7.4 Hz, 3H), 0.97 (d, J=6.9 Hz, 3H), 1.18-1.28 (m, 1H), 1.29-1.40 (m, 1H), 1.76-1.85 (m, 1H), 3.78 (s, 3H), 4.08 (d, J=3.8 Hz, 1H) ppm; ¹³C NMR (CDCl₃, 100 MHz) δ 175.6, 74.9, 52.5, 39.2, 23.9, 15.5, 11.9 ppm; LRMS (ESI-MS) calcd. for C₇H₁₄O₃Na [M+Na]⁺ 169, found 169.

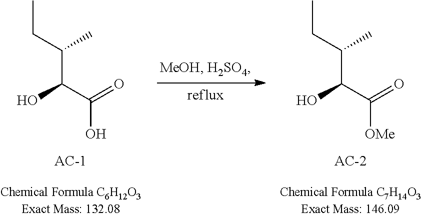

### 1-7. Intermediate Compound AC-3

To a solution of hydroxy ester compound AC-2 (8.21 g, 56.19 mmol) in pyridine (9.5 mL, 1.16 V) and dichloromethane (DCM)(9.5 mL, 1.16 V), tosyl chloride (p-TsCl) (16.06 g, 84.25 mmol, 1.5 equiv.) was added portion-wise at 0° C. to 4° C. Then the reaction mixture reacts at 19° C.-29° C. for 12-16 hours. The reaction was quenched by addition H₂O (33 mL, 4V). The organic layer was further washed with NaHCO₃ (sat. aq., 33 mL, 4V), and with H₂SO₄(aq. 1.0 M, 33 mL). The organic phase was dried (MgSO₄), filtered and concentrated at 30° C. to 35° C. The crude oil 17.46 g was used for next step without further purification. Compound AC-3: R_{f}0.70 (DCM); TLC stain: PMA; LRMS (ESI-MS) calcd. for C₁₄H₂₀O₅SNa [M+Na]⁺ 323, found 323.

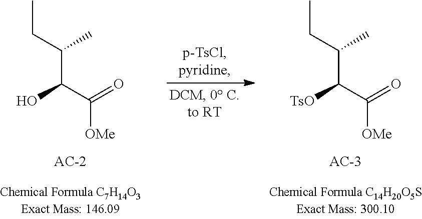

### 1-8. Intermediate Compound AC-4

A solution of ester compound AC-3 (16.70 g, 55.65 mmol) in THF-EtOH (1:1, 200 mL, 12 V) containing anhydrous LiCl (3.10 g, 73.22 mmol, 2.0 equiv.) was cooled to 0° C. and first portion of NaBH₄ (2.77 g, 73.22 mmol, 2.0 equiv.) was added at this temperature. The reaction mixture was stirred at 0° C. for 3.5 hours. Second portion of NaBH₄ (4.23 g, 111.30 mmol, 2.0 equiv.) was added at 0° C. and the reaction mixture was stirred at 0° C. for another 2 hours. Final portion of NaBH₄ (4.23 g, 111.30 mmol 2.0 equiv.) was added and stirred at 0° C. for 2 hours. The reaction was quenched by slowly addition of H₂SO₄ (1.0 M, 112.90 g) at 0° C. and diluted with H₂O (0.35 L) and NaHCO₃ (sat., aq. 5 mL g). The mixture was extracted with DCM (50 mL) twice. The organic layers were isolated and collected. Anhydrous MgSO₄ was added and the suspension was vigorously stirred and filtered through filter paper. Solvent was evaporated and crude product 12.70 g was obtained. Compound AC-4: R_{f}0.29 (DCM); TLC stain: PMA; ¹H NMR (400 MHz, CDCl₃) δ 0.81 (t, J=7.4 Hz, 3H), 0.85 (d, J=6.9 Hz, 3H), 1.02-1.13 (m, 1H), 1.36-1.46 (m, 1H), 1.68-1.78 (m, 1H), 2.45 (s, 3H), 3.72-3.80 (m, 2H), 4.50-4.54 (m, 1H), 7.35 (d, J=8.2 Hz, 2H), 7.82 (d, J=8.2 Hz 2H) ppm; ¹³C NMR (CDCl₃, 100 MHz) δ 145.0, 133.8, 130.0, 128.0, 88.7, 62.6, 36.5, 24.9, 21.8, 14.6, 11.4 ppm; LRMS (ESI-MS) calcd. for C₁₃H₂₀O₄SNa [M+Na]⁺ 295, found 295.

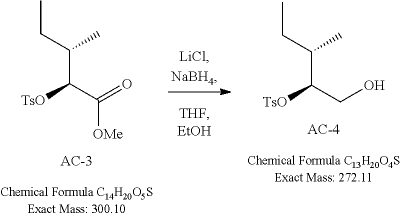

### 1-9. Intermediate Compound AC-5

A tosylate alcohol compound AC-4 (3.00 g, 11.03 mmol) was azeotropic distill with toluene twice. Crude compound AC-4 was diluted by THF (5 mL), and then DBU (5 mL) was added. The mixture was heated at 40° C. to 60° C. for 2.5 hours. Compound AC-4: R_{f}0.33 (DCM); TLC stain: PMA (currently, the TLC monitor was based on the starting material compound AC-4. Disappearance of compound AC-4 in the TLC suggested the reaction was fully converted.) Product was purified by distillation at 1 atm. external temperature between 64° C.-141° C. Epoxide compound AC-5 was collected at steam temperature between 38° C.-78° C. to give compound AC-5 in THF, which was directly used to the next step; ¹H NMR (400 MHz, CDCl₃) δ 0.88 (t, J=7.6 Hz, 3H), 0.99 (d, J=6.4 Hz, 3H), 1.10-1.46 (m, 3H), 2.50 (dd, J=2.8 Hz, J=5.0 Hz, 1H), 2.63-2.67 (m, 1H), 2.73 (dd, J=4.1 Hz, J=4.8 Hz, 1H) ppm; ¹³C NMR (CDCl₃, 100 MHz) δ 57.1, 47.1, 38.0, 26.5, 16.9, 11.8 ppm.

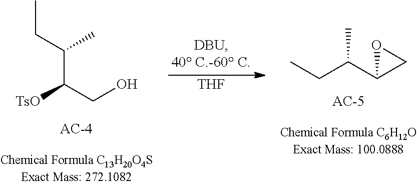

### 1-10. Intermediate Compound AC-6

The solution of 1,3-dithiane (2.95 g, 24.50 mmol, 2.2 equiv.) in tetrahydrofuran (THF) (29.5 mL, 10 V) was added with n-butyllithium (n-BuLi) (2.5 M, 13.72 mL, 34.29 mmol, 1.4 equiv.) at −40′C under nitrogen and the mixture was stirred for 2 h. A solution of compound AC-5 (theoretical amount: 1.10 g, 10.99 mmol) in tetrahydrofuran was slowly added to the reaction mixture over a period of 12 min at −40° C., and stirred for additional 4 h at −40° C. Then, the reaction temperature was warmed to 0° C. and stirred for another 13 h. The reaction mixture was acidified by adding H₂SO₄ (2.0 N, 17.29 g) until pH=5, and then neutralized by saturated NaHCO_{3(aq.)} to pH=6-7. The reaction mixture was extracted with EtOAc (2×10.0 mL). The combined organic layers were dried over MgSO₄ and concentrated to give crude compound AC-6: R_{f}0.67 (EtOAc/Hexane 1/5); TLC stain: PMA. The residue was purified by silica gel-column chromatography (silica gel, hexane/EtOAc=5:1) to give compound AC-6 (1.01 g, 36% four steps from compound AC-2) as colorless oil; ¹H NMR (400 MHz, CDCl₃) δ 0.87 (d, J=6.8 Hz, 3H), 0.89 (t, J=7.3 Hz, 3H), 1.11-1.21 (m, 1H), 1.33-1.52 (m, 2H), 1.75-1.92 (m, 3H), 2.07-2.15 (m, 1H), 2.79-2.95 (m, 4H), 3.81-3.85 (m, 1H), 4.24 (dd, J=5.0 Hz, J=9.4 Hz, 1H) ppm; ¹³C NMR (CDCl₃, 100 MHz) δ 71.6, 44.8, 40.5, 40.0, 30.5, 30.2, 26.1, 25.8, 13.6, 11.8 ppm; LRMS (ESI-MS) calcd. for C₁₀H₂₀OS₂Na [M+Na]⁺ 243, found 243.

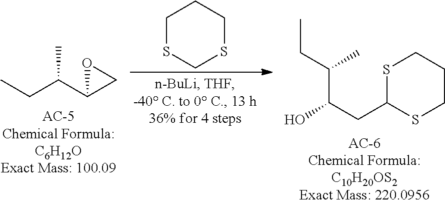

### 1-11. Intermediate Compound AC-7

Compound AC-6 (1.0 g, 4.54 mmol) was azeotropic distillation with toluene (2×10 mL). Compound AC-6 was dissolved in dried THF (7.7 mL) and DMF (3.8 mL) under ice bath, and then BnBr (3.63 mL, 30.44 mmol) and lithium hexamethyldisilazide (LiHMDS) (1 M in THF, 6.82 mL, 6.82 mmol) was added to reaction mixture. The resulting mixture was stirred under ice bath to room temperature for 2 h. 2.0 N H₂SO₄ (6.51 g) was slowly added until pH=6-7, diluted with 3 mL H₂O, and then the mixture was extracted by EtOAc (2×10 mL). The combined EtOAc layer was dried over MgSO₄ and concentrated by rotavapor at 35° C. Crude syrup was purified by column chromatography (silica gel, MP: EtOAc/Hexane=1/50 to 1/5) to get compound AC-7 (0.94 g, 67% yield) as a yellow syrup. R_{f} 0.45 (EtOAc/Hexane 1/10); TLC stain: PMA. ¹H NMR (400 MHz, CDCl₃) δ 7.38-7.26 (m, 5H), 4.60 (d, J=11.5 Hz, 1H), 4.52 (d, J=11.5 Hz, 1H), 4.12 (dd, J=9.9, 4.6 Hz, 1H), 3.65 (td, J=9.1, 3.7 Hz, 1H), 2.90-2.72 (m, 4H), 2.14-2.07 (m, 1H), 1.96-1.77 (m, 3H), 1.73-1.64 (m, 1H), 1.63-1.54 (m, 1H), 1.17-1.05 (m, 1H), 0.901 (d, J=6.9 Hz, 3H), 0.900 (t, J=7.4 Hz, 3H); ¹³C NMR (151 MHz, CDCl₃) δ 139.3, 128.4, 127.9, 127.8, 127.6, 79.7, 72.4, 44.7, 37.8, 37.0, 30.7, 30.2, 26.3, 24.7, 14.9, 12.3; LRMS (ESI-MS) calcd for C₁₇H₂₆OS₂Na [M+Na]⁺ 333, found 333.

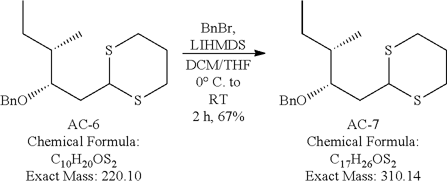 [SMILES: CC[C@H](C)[C@H](CC1SCCCS1)OCc1ccccc1 ; "BnBr,LIHMDS" ; CC[C@H](C)[C@H](CC1SCCCS1)OCc1ccccc1]

### 1-12. Intermediate Compound AC-8

Compound AC-7 (0.94 g, 3.03 mmol) was dissolved in ACN/H₂O (22.6 mL) solution at room temperature. K₂CO₃ (0.84 g, 6.06 mmol) and CH₃I (1.89 mL, 30.30 mmol) was added, and then the reaction mixture was warmed to 35° C. and stirred for 5 h. The mixture was diluted by H₂O (5 mL), neutralized by 2 N H₂SO₄ until pH 6, and then extracted by EA (2×5 mL). The combined EtOAc layer was dried over MgSO₄, concentrated by rotavapor, and purified by column chromatography (silica gel, M.P.: 1/20) to get the compound AC-8 (0.40 g, 60% yield) as a yellow liquid. R_{f}0.30 (EtOAc/Hexane 1/20); TLC stain: PMA. ¹H-NMR (400 MHz, CDCl₃) δ 9.79 (dd, J=2.5, 1.8 Hz, 1H), 7.36-7.27 (m, 5H), 4.57 (d, J=11.4 Hz, 1H), 4.51 (d, J=11.44 Hz, 1H), 3.92-3.88 (m, 1H), 2.66 (ddd, J=16.4, 8.5, 2.6 Hz, 1H), 2.49 (ddd, J=16.4, 3.9, 1.7 Hz, 1H), 1.77-1.68 (m, 1H), 1.65-1.57 (m, 1H), 1.16-1.04 (m, 1H), 0.918 (d, J=6.8 Hz, 3H), 0.917 (t, J=7.4 Hz, 3H); ¹³C NMR (151 MHz, CDCl₃) δ 202.2, 138.5, 128.5, 127.80, 127.76, 78.3, 71.9, 45.5, 38.1, 24.6, 15.0, 12.1; LRMS (ESI-MS) calcd. for C₁₇H₂₆OS₂Na [M+Na]⁺ 243, found N/A.

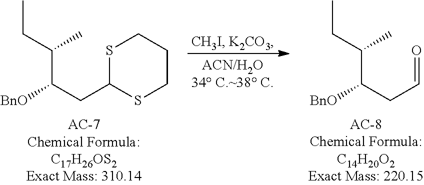 [SMILES: CC[C@H](C)[C@H](CC1SCCCS1)OCc1ccccc1 ; CC[C@H](C)[C@H](CC=O)OCc1ccccc1]

### 1-13. Intermediate Compound AC-9

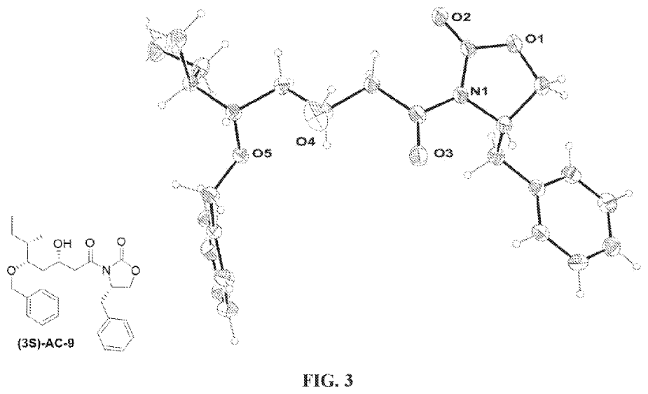

Aldehyde compound AC-8 (0.40 g, 1.80 mmol) and (S) Chiral Aux. (0.54 g, 1.80 mmol) were azeotropic distillation with toluene (2×10 mL). The mixture was dissolved in dried THF (4.4 mL), cold to −78° C., and then the solution was added to a deep blue solution of SmI₂ (0.1 M in THE 36 mL) at −78° C., stirred for 30 min. The reaction mixture warmed to room temperature, H₂O (6 mL) and 2 N H₂SO₄ (0.5 mL) was added, and then extracted by DCM (10 mL). Water layer was further extracted by EtOAc (3×10 mL). Organic layers were combined, dried over MgSO₄, and concentrated by rotavapor at 35° C. The residue was purified by column chromatography (silica gel, M.P.: EtOAc/Hexane=3/7) to get compound AC-9 (0.58 g, 73% yield) as a whit solid. As shown in FIG. 3, X-ray crystallography analysis of compound AC-9 confirmed the absolute configuration of compound AC-9, suggesting that the present synthetic route is feasible. R_{f}0.08 (EtOAc/Hexane 1/5); TLC stain: PMA; ¹H NMR (600 MHz, CDCl₃) δ 7.38-7.32 (m, 6H), 7.29-7.28 (m, 2H), 7.21-7.20 (m, 2H), 4.70-4.66 (m, 1H), 4.63 (d, J=11.3 Hz, 1H), 4.56 (d, J=11.3 Hz, 1H), 4.42-4.38 (m, 1H), 4.20-4.15 (m, 2H), 3.68-3.65 (m, 1H), 3.28 (dd, J=13.5, 3.3 Hz, 1H), 13.12 (dd, J=17.4, 3.3 Hz, 1H), 3.06 (dd, J=17.2, 8.8 Hz, 1H), 2.78 (dd, J=13.4, 9.5 Hz, 1H), 1.78-1.73 (m, 1H), 1.68-1.66 (m, 2H), 1.65-1.60 (m, 1H), 1.15-1.07 (m, 1H), 0.931 (d, J=6.9 Hz, 3H), 0.926 (t, J=7.0 Hz, 3H); ¹³C NMR (151 MHz, CDCl₃) δ 172.7, 153.5, 139.0, 135.3, 129.5, 129.1, 128.5, 128.0, 127.7, 127.6, 80.3, 72.3, 66.4, 65.3, 55.2, 43.2, 38.0, 37.6, 37.2, 24.4, 15.3, 12.3 ppm.

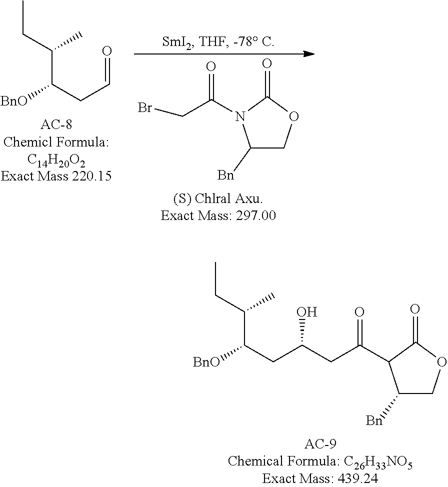 [SMILES: CC[C@H](C)[C@H](CC=O)OCc1ccccc1 ; O=C(CBr)N1C(=O)OCC1Cc1ccccc1 ; CC[C@H](C)[C@H](C[C@H](O)CC(=O)C1C(=O)OC[C@@H]1Cc1ccccc1)OCc1ccccc1]

### 1-14. Intermediate Compound AC-10

Compound AC-9 (50 mg, 0.11 mmol) was dissolved in THF at room temperature. N-methylimidazole (54 μL, 0.68 mmol) and iodine (0.17 g, 0.68 mmol), and then TBSCl (61 mg, 0.40 mmol) was added to reaction bottle. Reaction mixture was stirred at room temperature for 15 h, followed by addition of saturated Na₂S₂O_{3(aq)} (4 mL). The resulting mixture was extracted by EtOAc (2×4 mL), and organic layer was collected, dried over MgSO₄, and then concentrated by rotavapor as 35° C. Residue was purified by column chromatography (silica gel, M.P.: EtOAc/Hexane=1/10 to 1/5) to get compound AC-10 (49 mg, 78% yield) as a brown liquid. R_{f} 0.24 (EtOAc/Hexane 3/7, run for two times), TLC stain. PMA; ¹H NMR (600 MHz, CD₂Cl₂) δ 7.36-7.31 (m, 6H), 7.28-7.24 (m, 2H), 7.21-7.20 (m, 2H), 4.65-4.61 (m, 1H), 4.58 (d, J=11.3 Hz, 1H), 4.46 (d, J=11.4 Hz, 1H), 4.44-4.41 (m, 1H), 4.12 (s, 1H), 4.11 (d, J=1.1 Hz, 1H), 3.54 (td, J=8.6, 3.4 Hz, 1H), 3.32 (dd, J=16.1, 6.4 Hz, 1H), 3.23 (dd, J=13.4, 3.3 Hz, 1H), 2.99 (dd, J=16.3, 5.9 Hz, 1H), 2.78 (dd. J=13.4, 9.3 Hz, 1H), 1.80-1.58 (m, 4H), 1.13-1.06 (m, 1H), 0.912 (t, J=7.4 Hz, 3H), 0.908 (d, J=6.9 Hz, 3H), 0.88 (s, 9H), 0.10 (s, 3H), 0.06 (s, 3H); ¹³C NMR (151 MHz, CD₂Cl₂) δ 171.3, 153.7, 139.9, 136.0, 129.9, 129.2, 128.6, 127.9, 127.62, 127.57, 80.8, 71.3, 67.6, 66.5, 55.5, 44.2, 39.3, 38.1, 37.4, 26.1, 24.5, 18.3, 15.1, 12.5, −4.1, −4.4 ppm; LRMS (ESI-MS) calcd. for C₃₂H₄₇NO₅SiNa [M−Na]⁺ 576, found 576.

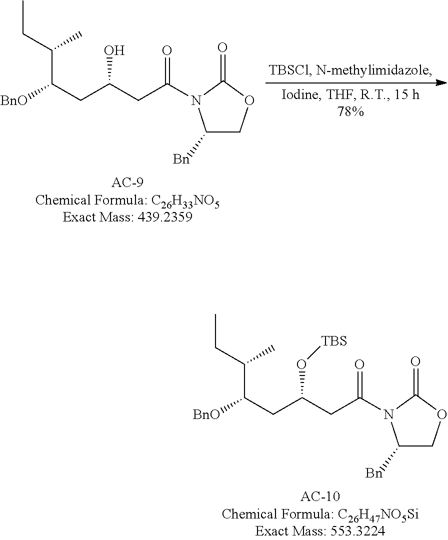

### 1-15. Intermediate Compound AC-11

Compound AC-10 (46 mg, 0.083 mmol) and Pd/C (10%, 92 mg) were suspended in THF. Reaction bottle was degassed and refilled by H₂ for 10 times, and then stirred under H₂ atmosphere (balloon) at R.T. for 3 h. The resulting mixture was filtered by a short pad of Celite®, washed by MeOH, filtrate was collected and concentrated by rotavapor at 35° C. Residue was purified by column chromatography (silica gel, EtOAc/Hex, M.P.: 1/10 to 1/5) to get compound AC-11 (39 mg, 99% yield) a colorless syrup. R_{f}0.21 (EtOAc/Hexane 1/5); TLC stain: PMA; ¹H NMR (600 MHz, CD₃OD) δ 7.33-7.31 (m, 2H), 7.27-7.22 (m, 3H), 4.71 (td, J=12.2, 3.0 Hz, 1H), 4.51-4.47 (m, 1H), 4.29-4.26 (m, 1H), 4.23 (dd, J=9.1, 2.8 Hz, 1H), 3.75 (td, J=9.0, 3.6 Hz, 1H), 3.40 (dd. J=15.4, 6.2 Hz, 1H), 3.16 (dd, J=13.5, 3.2 Hz, 1H), 2.95 (dd, J=15.3, 6.5 Hz, 1H), 2.93 (dd, J=13.3, 8.3 Hz, 1H), 1.65-1.57 (m, 2H), 1.56-1.50 (m, 1H), 1.39-1.32 (m, 1H), 1.20-1.13 (m, 1H), 0.93 (t, J=7.5 Hz, 3H), 0.90-0.88 (m, 12H), 0.15 (s, 3H), 0.11 (s, 3H); I³C NMR (151 MHz, CD₃OD) δ 172.5, 166.0, 155.3, 136.9, 130.6, 129.8, 128.2, 71.8, 68.3, 67.5, 56.5, 44.9, 43.0, 42.4, 38.4, 26.7, 26.4, 18.9, 17.1, 14.3, 12.3, −4.2 ppm; LRMS (ESI-MS) calcd. for C₂₅H₄₁NO₅SiNa [M+Na]⁺ 486. found 486.

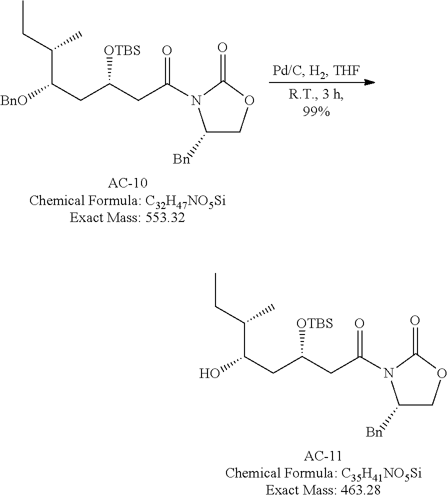 [SMILES: CC[C@H](C)[C@H](C[C@@H](CC(=O)N1C(=O)OC[C@@H]1Cc1ccccc1)O[Si](C)(C)C(C)(C)C)OCc1ccccc1 ; CC[C@H](C)[C@@H](O)C[C@@H](CC(=O)N1C(=O)OC[C@@H]1Cc1ccccc1)O[Si](C)(C)C(C)(C)C]

### 1-16. Intermediate Compound AC-12

To 2-neck flask, donor compound Ara. 2 (0.1743 g, 0.35 mmol), diphenyl sulfoxide (Ph₂SO) (0.2085 g, 1.02 mmol) and 2,4,6-tri-t-butyl pyridine (0.3442 g, 1.36 mmol) were added. Moisture in the mixture was removed by toluene distillation 3 times. Then flame-dried 4 Å molecular sieve, DCM (2.8 mL) and toluene (20 mL) were added and stirred at room temperature for 60 min. At −78° C., Tf₂O (0.085 mL, 0.51 mmol) was added and stirred for 30 min. The reaction was warm-up to −50° C. and stirred for 4 hours. At −77° C., a compound AC-11 solution composed of compound AC-11 (0.0523 g, 0.11 mmol), DCM (1.0 mL) and toluene (5.0 mL) was added. Then the reaction mixture was stirred at room temperature overnight (about 14 h). The reaction was quenched with triethylamine (0.2 mL, 2.29 mmol), and filtered through Celite®. The filtrate was concentrated at 35° C. to afford 1.1396 g crude. A 25 g normal phase silica gel column and eluent of 5% EtOAc/hexane were used to purify the crude. Product-compound AC-12 (0.1044 g, 99%) was obtained. Compound AC-12: R_{f}0.39 (EtOAc/hexanes=1/9); TLC stain: p-anisaldehyde.

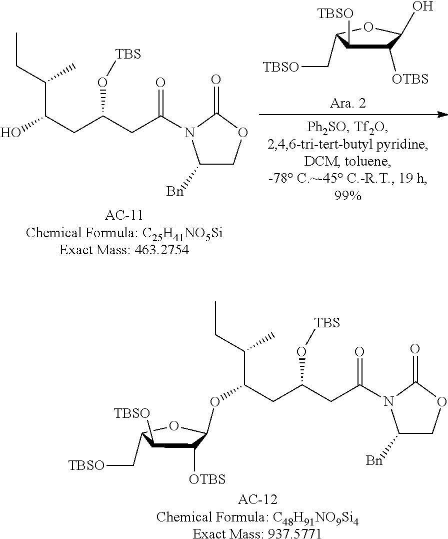

### 1-17. Intermediate Compound AC-13

Compound AC-12 (0.1018 g, 0.11 mmol, 1.0 equiv.) was diluted with THF (2.0 mL, 20 V) and H₂O (0.50 mL, 5 V) in flask. After cooled by ice-bath, LiOH monohydrate (0.0272 g, 0.65 mmol, 6.0 equiv.) and H₂O₂ (0.089 g, 0.75 mmol, 6.9 equiv.) were added. The reaction was stirring at 25° C. for 110 min. The reaction was quenched by 1N HCl to pH 6-7. The 0.1188 g of crude compound AC-13: R_{f} 0.25 (EtOAc/hexane=1/9; TLC stain: p-anisaldehyde) was obtained after extraction with EtOAc and dry over MgSO₄. The crude was purified by a 24 g normal phase silica gel column and eluent from 5% to 10% EtOAc/hexane. The fractions containing product were combined, concentrated and dried by vacuum to afford compound AC-13 (0.0537 g, 64%). ¹H NMR (600 MHz, CDCl₃) δ 4.92 (s, 1H), 4.25-4.21 (m, 1H), 3.99-3.89 (m, 1H), 3.96-3.99 (m, 1H), 3.95 (s, 1H), 3.68 (dd, J=10.5, 4.9 Hz, 1H), 3.64 (dd, J=10.4, 6.2 Hz, 1H), 3.60 (td, J=8.9, 2.9 Hz, 1H), 2.68 (dd, J=15.3, 4.7 Hz, 1H), 2.48 (dd, J=15.4, 5.0 Hz, 1H), 1.67 (ddd, J=14.5, 6.4, 2.8 H, 1H), 1.64-1.57 (m, 2H), 1.56-1.50 (m, 1H), 1.03-0.98 (m, 1H), 0.90-0.87 (m, 41H), 0.83 (d, J=6.8 Hz, 3H), 0.157 (s, 3H), 0.121 (s, 3H), 0.080 (s, 3H), 0.077 (s, 3H), 0.064-0.058 (m, 12H); ¹³C NMR (151 MHz, CDCl₃) δ 173.34, 107.29, 87.08, 83.98, 79.48, 77.91, 68.54, 63.76, 42.96, 38.61, 36.98, 26.13, 25.89, 23.63, 18.58, 18.09, 18.01, 15.05, 12.33, 4.27, −4.42, −4.48, −4.61 ppm.

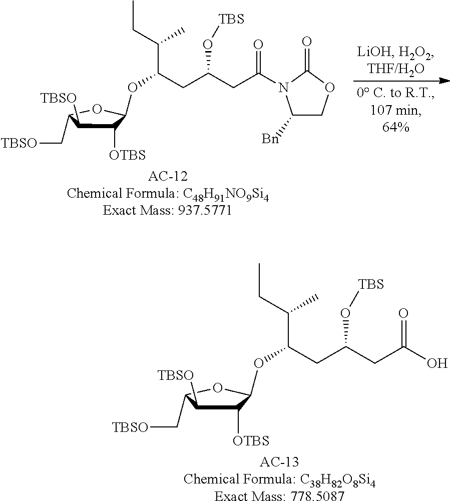

### 1-18. Intermediate Compound AC-14

Compound AC-13 (0.010 g, 0.01 mmol, 1.0 equiv.) was diluted with toluene (0.4 mL, 41 V). Triethylamine (ET₃N) (0.002 mL, 0.015 mmol, 1.2 equiv.) and 2,4,6-trichlorobenzoyl chloride (0.002 mL, 0.014 mmol, 1.1 equiv.) were added at room temperature. The mixture was stirred for 30 min at room temperature. Compound AC-17 (0.0049 g, 0.015 mmol, 1.2 equiv.) was diluted with toluene (0.14 mL, 14V) and added to reaction mixture. Addition of DMAP (0.002 g, 0.018 mmol, 1.4 equiv.) was followed. The reaction was stirred at room temperature for 45 min and work up by concentration. The crude compound AC-14: R_{f}0.80 (EtOAc/hexane=1/3; TLC stain: p-anisaldehyde) was purified by 25 g normal phase silica gel column eluted with hexanes, 2% EtOAc/hexanes and 5% EtOAc/hexanes to afford compound AC-14 (0.0082 g, 59%). Compound AC-13: R_{f} 0.67; compound AC-17: R_{f} 0.48 (EtOAc/hexanes=1/3; TLC stain: p-anisaldehyde); ¹H NMR (600 MHz, CDCl₃) δ 4.90-4.88 (m, 2H), 4.20-4.16 (m, 1H), 4.11-4.07 (m, 1H), 4.03 (dd, J=3.9, 2.1 Hz, 1H), 3.95-3.93 (m, 2H), 3.95-3.93 (m, 2H), 3.70-3.66 (m, 2H), 3.65 (s, 3H), 3.59-3.57 (m, 1H), 2.52-2.41 (m, 4H), 1.78-1.48 (m, 7H), 1.45-1.40 (m, 1H), 1.15-0.96 (m, 2H), 0.90-0.82 (m, 61H), 0.105 (s, 3H), 0.07-0.06 (m, 24H), 0.03 (s, 3H); ¹³C NMR (151 MHz, CDCl₃) δ 171.83, 171.07, 107.35, 86.05, 84.23, 79.24, 77.59, 74.37, 67.31, 67.06, 63.25, 51.56, 43.65, 43.20, 39.44, 38.34, 37.00, 26.12, 26.04, 25.92, 25.90, 25.33, 24.09, 18.55, 18.09, 18.04, 18.02, 14.71, 14.26, 12.34, 12.09, −4.13, −4.27, −4.38, −4.44, −4.49, −4.54, −4.58 ppm; LRMS (ESI-MS) calcd. for C₅₄H₁₁₈NO₁₁Si₅ [M+NH₄]⁺ 1096.76. found 1096.85.

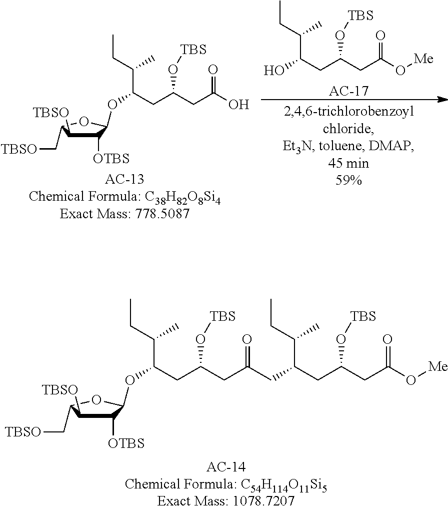

### 1-19. Final Product Compound AC-15

Methyl ester compound AC-14 (0.0082 g, 0.0076 mmol), barium hydroxide octahydrate (Ba(OH)₂8H₂O) (0.6 g, 1.898 mmol, 250 equiv.) and methanol (MeOH) (1 mL, 96V) were mixed and stirred at room temperature for 21 hours. The reaction was quenched by addition of HCl (1.0 N, 3.5 mL) to pH 3-4. The resulting mixture was extracted with EtOAc and DCM. The organic layers were combined, dried over MgSO₄ and concentrated to afford the crude compound AC-15. The crude compound AC-15: R_{f} 0.08 (EtOAc/hexane=1/9; TLC stain: p-anisaldehyde) was purified by 25 g normal phase silica gel column eluted with 5% EtOAc/hexane and 10% EtOAc/hexane to afford compound AC-15 (0.0029 g, 92% yield) and compound AC-14 (0.0050 g, 39% conversion). Compound AC-14: R_{f} 0.57 (EtOAc/hexane=1/9, TLC stain: p-anisaldehyde); ¹H NMR (600 MHz, CDCl₃) δ 4.92-4.88 (m, 2H), 4.19-4.14 (m, 2H), 4.01 (dd, J=3.6 2.0 Hz, 1H), 4.00-3.96 (m, 2H), 3.72-3.66 (m, 2H), 3.61-3.57 (m, 1H), 2.57 (dd, J=15.2, 5.2 Hz, 1H), 2.51 (dd, J=15.2, 6.2 Hz, 1H), 2.47 (dd, J=15.2, 5.0 Hz, 1H), 2.43 (dd, J=15.1, 6.1 Hz, 1H), 1.80-1.50 (m, 7H), 1.48-1.39 (m, 1H), 1.34-1.24 (m, 1H), 1.16-0.98 (m, 2H), 0.98-0.80 (m, 61H), 0.12-0.06 (m, 30H); ¹³C NMR (151 MHz, CDCl₃) δ 172.30, 171.35, 107.45, 86.32, 83.95, 79.33, 77.99, 74.75, 67.69, 67.54, 63.52, 43.62, 42.84, 39.65, 39.16, 38.88, 37.00, 26.13, 26.02, 25.97, 25.90, 25.68, 25.27, 23.85, 18.60, 18.09, 18.02, 17.97, 14.85, 14.33, 12.32, 12.05, −4.17, −4.27, −4.42, −4.49, −4.58; LRMS (ESI-MS) calcd. for C₅₃H₁₁₆NO₁₁Si₅ [M+NH₄]⁺ 1082.74, found 1082.81.

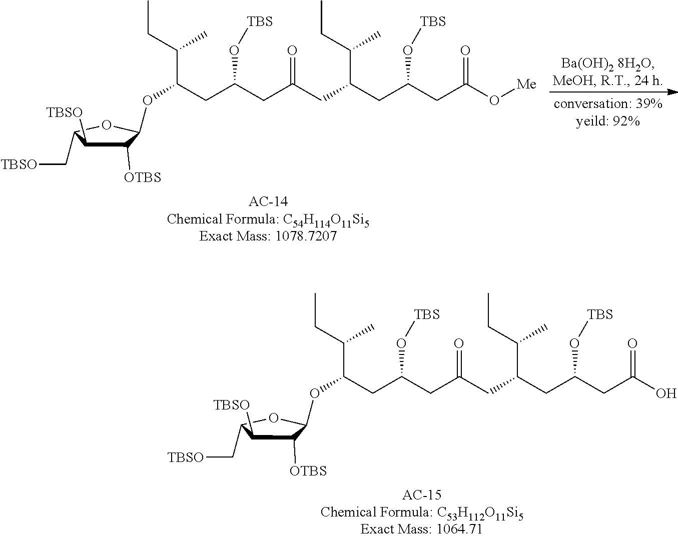

### Example 2: Isoleucine Synthesis Thesis

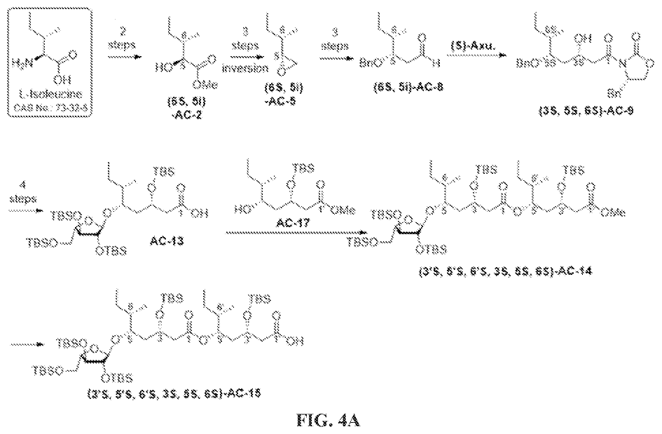

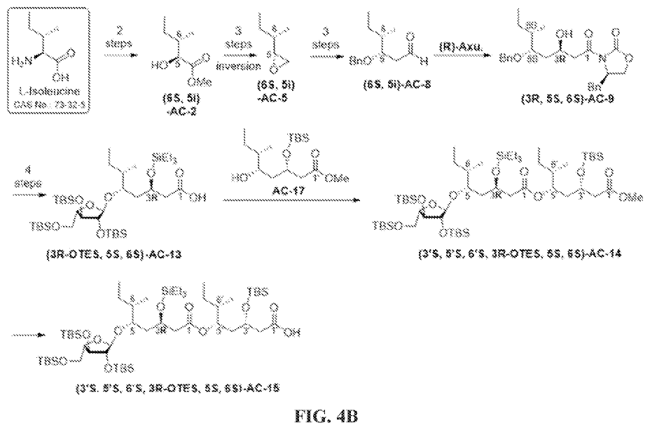

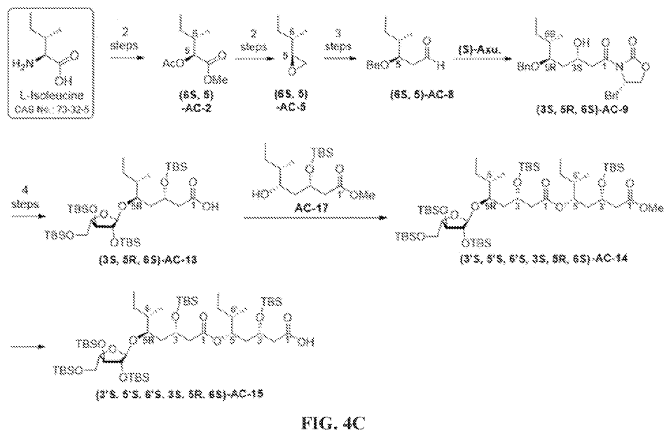

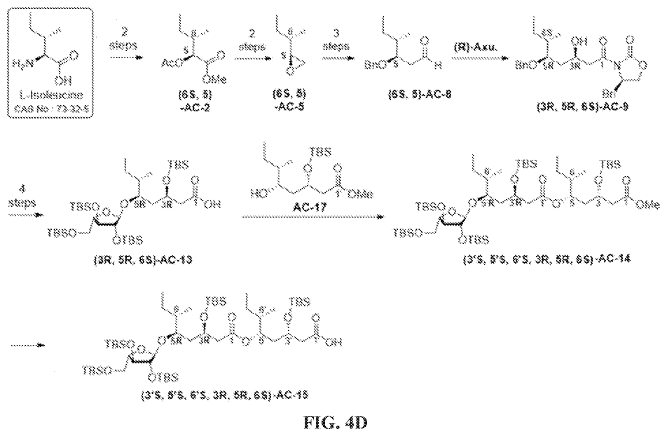

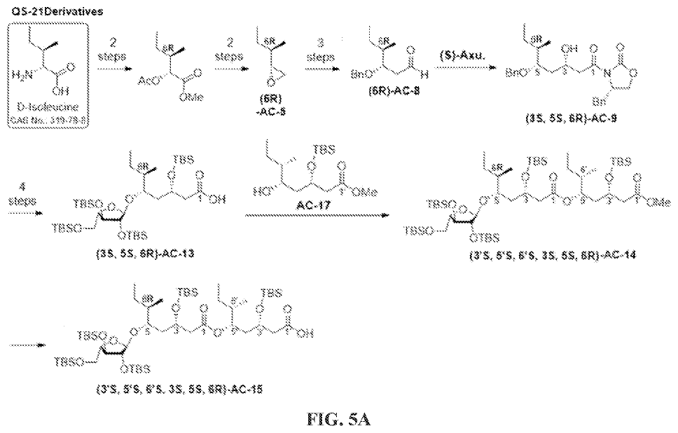

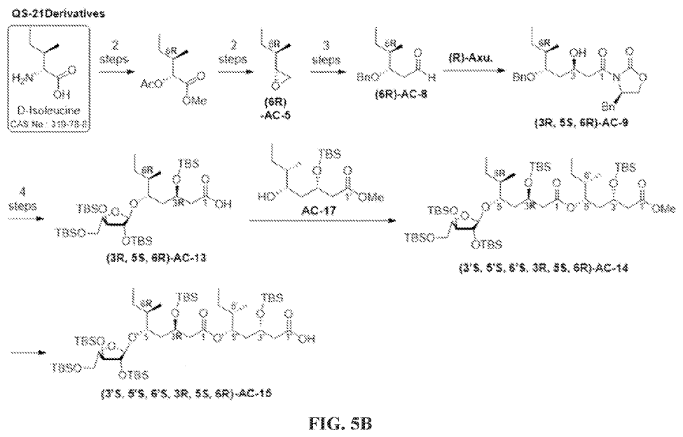

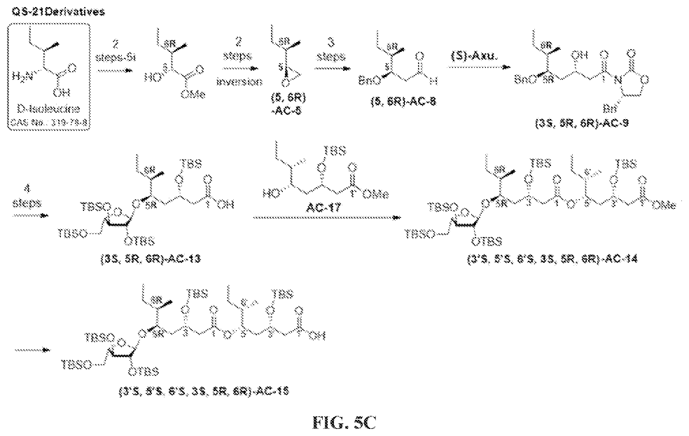

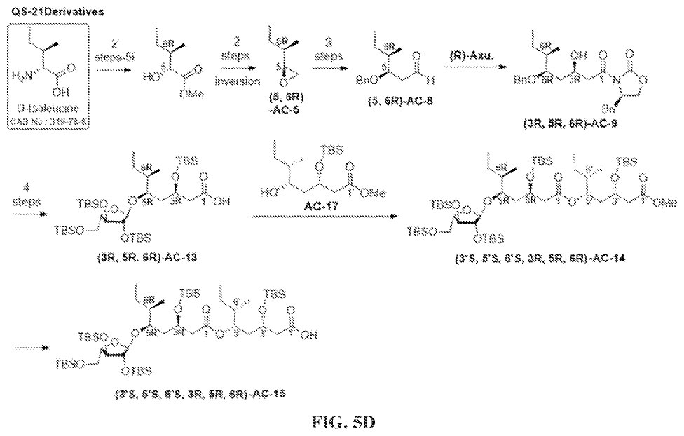

The method using isoleucine to get acyl chain can apply to inverse each of six stereocenters (3, 5, 6, 3′, 5′ and 6′) of compound AC-15 independently. FIGS. 4A-4D and 5A-5D demonstrate the way to get 8 outcomes from controlling stereocenters 3, 5 and 6, but 3′, 5, and 6′ stay in S-form. Totally, 64 combinations can be produced by controlling six stereocenters. Stereochemistry on 3S and 3R can be controlled by (S)-Axu. or (R)-Axu. In the present case. (S)-Axu. creates compound (3S)-AC-9 and (R)-Axu. results in compound (3R)-AC-9. Stereochemistry on 5S and 5R uses different synthetic methods to control. Inversion process from compound (6S, 5i)-AC-2 to compound (6S, 5i)-AC-5 changes stereochemistry from L-isoleucine to get compound (3′S, 5′S, 6′S, 3S, 5S, 6S)-AC-15 in the end. On the other hand, no inversion process keeps stereochemistry from L-isoleucine and gets compound (3′S, 5′S, 6′S, 3S, 5R, 6S)-AC-15. Stereochemistry on 6S and 6R is determined by the chiral center on L-isoleucine or D-isoleucine. Nature form QS-21 uses compound (3S, 5S, 6S)-AC-9 as intermediate, whose stereochemistry is from L-isoleucine. In contrast, compound (3S, 5S, 6R)-AC-9 is from L-isoleucine.

### 2-1. Synthesis of Compound (3R)-AC-9

Aldehyde compound AC-8 (0.23 g, 1.06 mmol) and (R)-Aux. (0.31 g, 1.06 mmol, 1.0 equiv.) were azeotropic distillation with toluene (2×10 mL). The mixture was dissolved in dried THF (1.8 mL) and added to deep blue solution of SmI₂ (0.1 M in THF, 21 mL, 2.0 equiv.) at −78° C. The reaction kept stir for 1 h in −78° C. The reaction mixture was quenched with H₂SO₄ (2 N, 0.5 mL) at room temperature. Water layer was further extracted by EtOAc (3 times). Organic layers were combined, dried over MgSO₄ and concentrated by rotavapor at 35° C. to offer crude compound (3R)-AC-9 (0.56 g). The residue was purified by column chromatography (10 g silica gel, mobile phase: EtOAc/Hex=3/7) to get compound (3R)-AC-9 (0.23 g, 49% yield) as a whit solid. Compound (3R)-AC-9: R_{f}0.06, compound AC-8: R_{f}0.57 (EtOAc/Hexane 1/4); TLC stain: PMA; ¹H NMR (600 MHz, CDCl₃) δ 7.35-7.31 (m, 6H), 7.29-7.25 (m, 2H), 7.22-7.21 (m, 2H), 4.70-4.66 (m, 1H, H-16), 4.63 (d, J=11.3 Hz, 1H, H-10), 4.48 (d, J=11.3 Hz, 1H, H-10), 4.36 (tt, J=13.1, 3.5 Hz, 1H, H-3), 4.19-4.14 (m, 2H, H-17), 3.65 (dt, J=9.4, 3.6 Hz, 1H, H-5), 3.30 (dd, J=13.4, 3.3 Hz, 1H, H-18) 3.13 (dd, J=16.7, 8.7 Hz, 1H, H-2), 3.03 (dd, J=16.8, 3.5 Hz, 1H, H-2), 2.76 (dd. J=13.4, 9.7 Hz, 1H, H-18), 1.85-1.76 (m, 2H, H-4.6), 1.70-1.63 (m, 2H, H-4, 7), 1.15-1.08 (m, 1H, H-7), 0.93 (t, J=7.4 Hz, 3H, H-8), 0.91 (d, J=6.8 Hz, 3H, H-9); ¹³C NMR (151 MHz, CDCl₃) δ 171.8 (C-1), 153.6 (C-15), 138.4, 135.4, 129.6, 129.1, 128.6, 127.9, 127.8, 127.5, 83.4 (C-5), 71.2 (C-10), 67.7 (C-3), 66.4 (C-17), 55.3 (C-16), 43.2 (C-2), 38.1 (C-18), 36.7 (C-6), 36.3 (C₄), 24.0 (C-7), 15.3 (C-9), 12.5 (C-8) ppm; LRMS (ESI-MS) calcd. for C₂₆H₃₃O₅NNa [M+Na]⁺ 462, found 462.

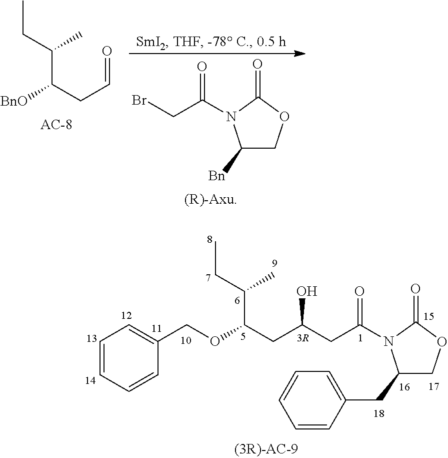

### 2-2. Synthesis of Compound (3R-OTES, 5S, 6S)-AC-10

Compound (3R)-AC-9 (0.23 g, 0.53 mmol) was azeotropic distillation with toluene. Then imidazole (0.30 g, 4.38 mmol, 8.2 equiv.) and DMAP (0.05 g, 0.37 mmol, 0.7 equiv.) were added to reaction bottle. The mixture was diluted with DMF (4.0 mL). In ice-bath, TESOTf (0.50 mL, 2.19 mmol, 4.1 equiv.) was added. The reaction was stirred at room temperature for 2.5 h. Reaction was quenched by addition of H₂O (2 mL) and extracted with DCM (5 mL) twice. The organic layers were combined, dried over MgSO₄ and then concentrated by rotavapor as 35° C. to provide crude (3.04 g). Crude was purified by column chromatography (22 g silica gel, mobile phase: EtOAc/Hexane=0 to 1/9) to get compound (3R-OTES, 5S, 6S)-AC-10 (0.29 g, >99% yield) as a colorless liquid. Compound (3R-OTES, 5S, 6S)-AC-10: R_{f}0.57 (EtOAc/Hexane 1/4); TLC stain: PMA; ¹H NMR (400 MHz, CDCl₃) δ 7.38-7.24 (m, 8H), 7.17-7.15 (m, 2H), 4.60-4.51 (m, 2H), 4.51-4.43 (m, 2H), 4.13-4.05 (m, 1H), 4.01 (dd, J=9.1, 2.9 Hz, 1H), 3.52-3.43 (m, 1H), 3.26-3.15 (m, 2H), 3.11 (dd, J=16.5, 6.0 Hz, 1H), 2.35 (dd, J=13.3, 10.2 Hz, 1H), 1.88-1.51 (m, 4H), 1.19-1.03 (m, 1H), 1.01-0.86 (m, 15H), 0.66-0.53 (m, 6H) ppm; LRMS (ESI-MS) calcd. for C₃₂H₄₇O₅NSiNa [M+Na]⁺ 576, found 576.

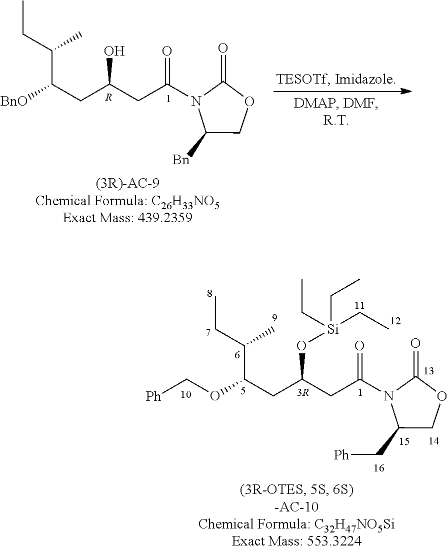

### 2-3. Synthesis of Compound (3R-OTES, 5S, 6S)-AC-11

Compound (3R-OTES, 5S, 6S)-AC-10 (0.29 g, 0.52 mmol) was diluted by THF (2.8 mL, 10 V). Pd/C (10%, 0.57 g, 2 W) was added to reaction. The reaction flask was degassed and filled with hydrogen (H₂) for 10 times. Hydrogenation was carried out with strong stirring for 2.5 hours at room temperature under balloon pressure. The reaction mixture was filtered through Celite® pad, which was washed with EtOAc. The filtrate was concentrated at 35° C. to afford crude compound (3R-OTES, 5S, 6S)-AC-11 (0.22 g). The crude was purified by 11 g normal phase silica gel column which eluted with 10%-20% EtOAc/hexane to get compound (3R-OTES, 5S, 6S)-AC-11 (0.17 g, 71%) as colorless oil. Compound (3R-OTES, 5S, 6S)-AC-11: R_{f} 0.37; compound (3R-OTES, 5S, 6S)-AC-10: R_{f}0.60 (EtOAc/Hexane 1/4); TLC stain: PMA; ¹H NMR (600 MHz, MeOD) δ 7.33-7.31 (m, 2H), 7.27-7.23 (m, 3H), 4.71 (tt, J=8.1, 3.2 Hz, 1H), 4.53-4.49 (m, 1H), 4.27 (t, J=8.5 Hz, 1H), 4.22 (dd, J=9.0, 2.9 Hz, 1H), 3.70-3.67 (m, 1H), 3.27 (dd, J=16.0, 7.1 Hz, 1H), 3.17 (dd, J=13.5, 3.2 Hz, 1H), 3.12 (dd, =16.1, 5.1 Hz, 1H), 2.92 (dd, J=13.5, 8.4 Hz, 1H), 1.79-1.68 (m, 2H), 1.58-1.49 (m, 1H), 1.44-1.35 (m, 1H), 1.26-1.16 (m, 1H), 0.98 (t, J=8.0 Hz, 9H), 0.94 (t, J=7.4 Hz, 3H), 0.90 (d, J=6.8 Hz, 3H), 0.65 (q, J=7.9 Hz, 6H); ¹³C NMR (151 MHz, MeOD) δ 172.9, 155.4, 136.9, 130.6, 129.8, 128.2, 72.1, 68.9, 67.5, 56.5, 43.5, 43.3, 41.8, 38.5, 27.0, 13.8, 12.3, 7.2, 6.0 ppm; LRMS (ESI-MS) calcd. for C₂₅H₄₁O₅NSiNa [M+Na]⁺ 486, found 486.

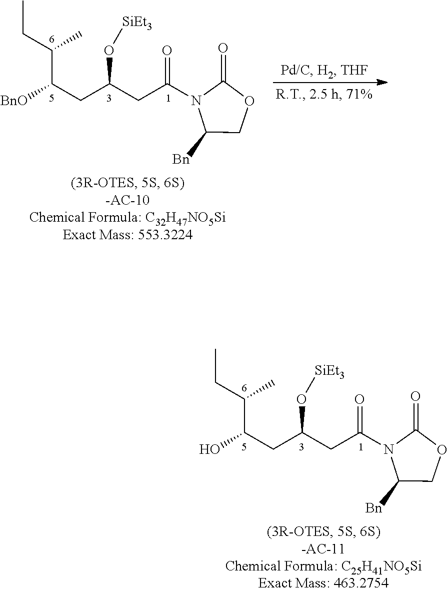

### 2-4. Synthesis of Compound (3R-OTES, 5S, 6S)-AC-12

To 2-neck flask, compound Ara. 2 (0.06 g, 0.13 mmol, 3.0 equiv.), diphenyl sulfoxide (Ph₂SO) (0.08 g, 0.39 mmol, 9.1 equiv.) and 2,4,6-tri-t-butyl pyridine (0.13 g, 0.52 mmol, 12.04 equiv.) were added. Moisture in the mixture was removed by toluene distillation 3 times. Then flame-dried 4 Å molecular sieve, DCM (2.8 mL) and toluene (20 mL) were added and stirred at room temperature for 60 min. At −78° C., Tf₂O (0.03 mL, 0.19 mmol, 4.50 equiv.) was added and stirred for 30 min. The reaction was warm-up to −50° C. and stirred for 4 hours. At −75° C. a solution which composed of compound (3R-OTES, 5S, 6S)-AC-11 (0.02 g, 0.04 mmol), DCM (0.4 mL) and toluene (3.6 mL) was added. Then the reaction mixture was stirred at room temperature overnight. The reaction was quenched with triethylamine (0.2 mL, 1.43 mmol, 33.0 equiv.). And filtration through Celite®. The filtrate was concentrated at 35° C. to afford 0.42 g crude. Crude was first purified with 11 g normal phase silica gel column and eluent of 5%-10% EtOAc/hexanes. Second purification was carried out with 10 g normal phase silica gel column and eluent of 5% EtOAc/hexanes. Product-compound (3R-OTES, 5S, 6S)-AC-12 (0.03 g, 67%. R_{f}0.58-0.54); compound (3R-OTES, 5S, 6S)-AC-11: R_{f} 0.07; compound Ara. 2: R_{f} 0.41 (EtOAc/hexanes=1/9). TLC stain: p-anisaldehyde: ¹H NMR (600 MHz, CDCl₃) δ 7.34 (t, J=7.5 Hz, 2H), 7.28 (d, J=7.4 Hz, 1H), 7.23 (d, J=7.1 Hz, 2H), 4.82 (s, 1H), 4.67-4.60 (m, 1H), 4.48-4.42 (m, 1H), 4.18-4.11 (m, 2H), 4.09 (dd, J=5.7, 2.8 Hz, 1H), 4.00 (d, J=2.7 Hz, 1H), 3.97 (dd, J=9.7, 4.2 Hz, 1H), 3.75 (dd, J=11.3, 4.4 Hz, 1H), 3.71 (dd, J=11.2, 3.9 Hz, 1H), 3.52 (dt, J=9.8, 2.8 Hz, 1H), 3.37 (dd, J=15.5, 8.1 Hz, 1H), 3.33 (dd, J=13.4, 3.2 Hz, 1H), 3.05 (dd, J=15.5, 3.6 Hz, 1H), 2.71 (dd, J=13.3, 9.9 Hz, 1H), 1.81-1.73 (m, 1H), 1.67-1.56 (m, 3H), 1.12-1.02 (m, 1H), 0.94 (t, J=8.0 Hz, 9H), 0.91-0.85 (m, 33H), 0.59 (q, J=8.0 Hz, 6H), 0.11-0.00 (m, 18H) ppm; LRMS (EST-MS) calcd. for C₄₈H₉₁O₉NSi₄Na [M+Na]⁺ 961, found 960. [M+Na]⁺ 961. found 960.

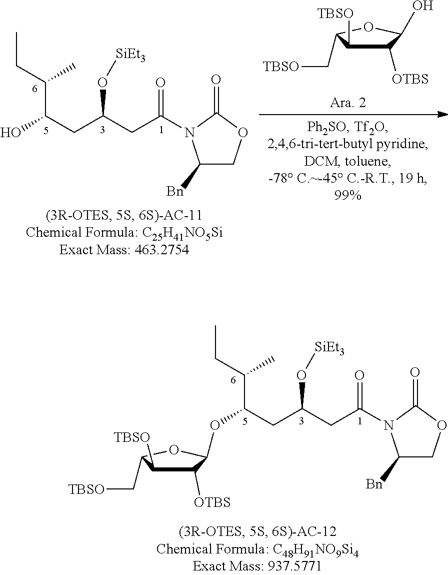

### 2-5. Synthesis of Compound (3R-OTES, 5S, 6S)-AC-13

Compound (3R-OTES, 5S, 6S)-AC-12 (0.0195 g, 0.021 mmol) was diluted with THF (0.39 mL, 20 V) and H₂O (0.1 mL, 5 V) in flask. After cooled by ice-bath, LiOH monohydrate (0.005 g, 0.124 mmol, 6.0 equiv.) and H₂O₂ (0.3 mL, 3.51 mmol, 168.5 equiv., 35%) were added. The reaction was stirring at 25° C. for 2 hours. The reaction was quenched by 1N HCl to pH 6-7. The 0.0233 g of crude was obtained after concentration at 35° C. The crude was purified by an 11 g normal phase silica gel column and eluent 10% EtOAc/hexanes. The fractions containing product were combined, concentrated and dried by vacuum to afford compound (3R-OTES, 5S, 6S)-AC-13-1 (0.0109 g, 67%, R_{f}0.35). Compound (3R-OTES, 5S, 6S)-AC-12: R_{f}0.42-0.46 (EtOAc/hexanes=1/9; TLC stain: p-anisaldehyde); compound (3R-OTES, 5S, 6S)-AC-13-1; ¹H NMR (600 MHz, CDCl₃) δ 4.84 (s, 1H), 4.35-4.30 (m, 1H), 4.01-3.96 (m, 2H), 3.88 (d, J=9.9, 5.0 Hz, 1H), 3.71-3.62 (m, 2H), 3.52 (dt, J=10.7, 2.4 Hz, 1H), 2.69 (dd, J=15.3.4.8 Hz, 1H), 2.63 (dd, J=15.3, 4.9 Hz, 1H), 1.78-1.71 (m, 1H), 1.64-1.55 (m, 3H), 1.03-0.98 (m, 1H), 0.97 (t, J=8.0 Hz, 9H), 0.93-0.81 (m, 33H), 0.64 (q, J=8.0 Hz, 6H), 0.11-0.04 (m, 18H); ¹³C NMR (151 MHz, CDCl₃) δ 173.0, 107.2, 103.8, 86.4, 85.7, 84.9, 84.8, 79.6, 79.3, 67.3, 63.1, 62.5, 40.3, 37.2, 29.8, 26.1, 25.90, 25.86, 23.5, 18.5, 18.1, 18.0, 15.1, 12.2, 6.8, 4.9, −4.1, −4.3, −4.5, −4.6 ppm; LRMS (ESI-MS) calcd. for C₃₈H₈₂O₈Si₄Na [M+Na]⁺ 801. found 801. Compound (3R-OTES, 5S, 6S)-AC-13-2; ¹H NMR (600 MHz, CDCl₃) δ 4.98 (d, J=3.8 Hz, 1H), 4.30-4.25 (m, 1H), 3.97 (t, J=4.5 Hz, 1H), 3.92 (dd, J=5.0, 3.8 Hz, 1H), 3.77-3.69 (m, 2H), 3.65-3.61 (m, 2H), 2.71 (dd, J=15.1, 6.5 Hz, 1H), 2.53 (dd, J=15.0, 4.8 Hz, 1H), 1.80-1.60 (m, 4H), 1.04-0.98 (m, 1H), 0.96 (t, J=8.0 Hz, 9H), 0.92-0.87 (m, 30H), 0.83 (d, J=7.0 Hz, 3H), 0.63 (q, J=7.9 Hz, 6H), 0.11 (s, 3H), 0.10 (s, 3H), 0.09 (s, 3H), 0.07 (s, 3H), 0.05 (s, 6H) ppm; LRMS (ESI-MS) calcd. for C₃₈H₈₂O₈Si₄Na [M+Na]⁺ 801, found 801.

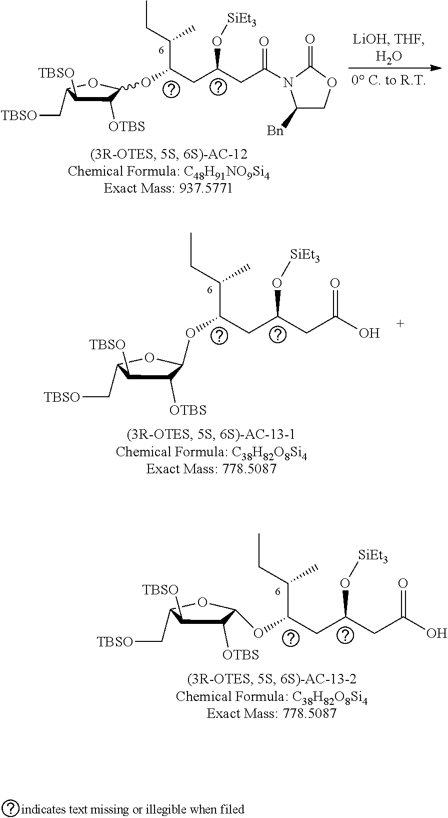

Unless defined otherwise, all technical and scientific terms and any acronyms used herein have the same meanings as commonly understood by one of ordinary skill in the art in the field of this invention. Although any compositions, methods, kits, and means for communicating information similar or equivalent to those described herein can be used to practice this invention, the preferred compositions, methods, kits, and means for communicating information are described herein.

What is claimed is:

1. A method of preparing a compound of Formula I, comprising: 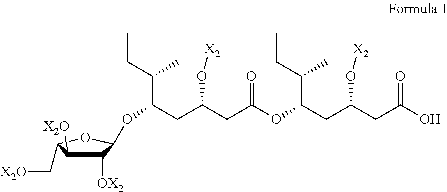
(a) performing hydroxylation and esterification of L-isoleucine to obtain compound AC-2; 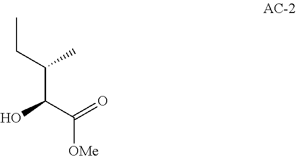
(b) treating compound AC-2 by intramolecular epoxidation and addition of 1,3-dithiane with an inversion process to obtain compound AC-6; 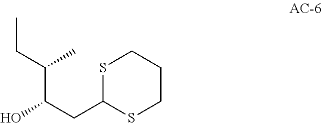
(c) installing a first alcohol protecting group to compound AC-6 and unmasking thio-protection to obtain Formula I-1 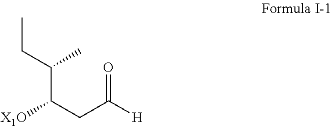
(d) performing an aldol reaction of Formula I-1 with compound (S)-Aux. by SmI₂ to obtain Formula I-2; 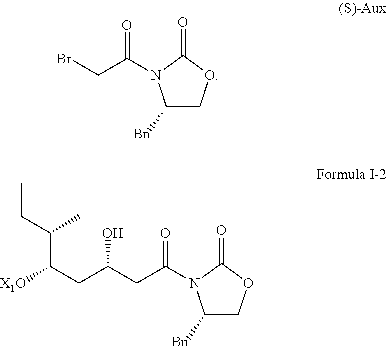
(e) installing a second alcohol protecting group to Formula I-2 to obtain Formula I-3; and 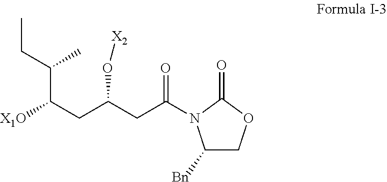
(f) removing the first alcohol protecting group from Formula I-3 via hydrogenolysis to obtain Formula I-4, 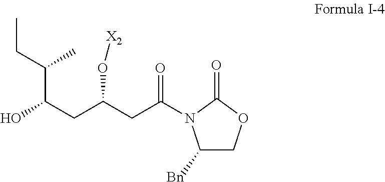
wherein X₁ is the first alcohol protecting group, and X₂ is the second alcohol protecting group.

2. The method of claim 1, wherein each X₁ is independently selected from a group consisting of methyl, benzyl (Bn), p-methoxybenzyl (PMB), methoxymethyl (MOM), methoxyethoxymethyl (MEM), trimethylsilyl (TMS), triethylsilyl (TES), triisopropylsilyl (TIPS), tert-butyldimethylsilyl (TBDMS), tert-butyldiphenylsilyl (TBS/TBDPS) and tetrahydropyranyl (THP).

3. The method of claim 1, wherein each X₂ is independently selected from a group consisting of methyl, benzyl (Bn), p-methoxybenzyl (PMB), methoxymethyl (MOM), methoxyethoxymethyl (MEM), trimethylsilyl (TMS), triethylsilyl (TES), triisopropylsilyl (TIPS), tert-butyldimethylsilyl (TBDMS), tert-butyldiphenylsilyl (TBS/TBDPS) and tetrahydropyranyl (THP).

4. The method of claim 1, further comprising:
(g) performing glycosylation of Formula I-4 with Formula I-a to obtain Formula I-5 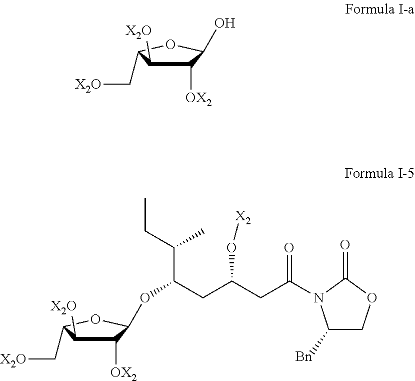
(h) treating Formula I-5 with lithium hydroxide and hydrogen peroxide to obtain Formula I-6; 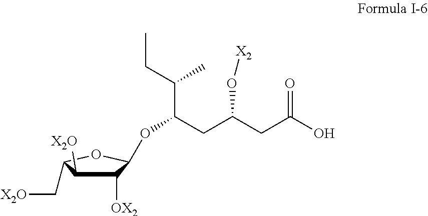
(i) coupling Formula I-6 with Formula I-b to obtain Formula I-7; and 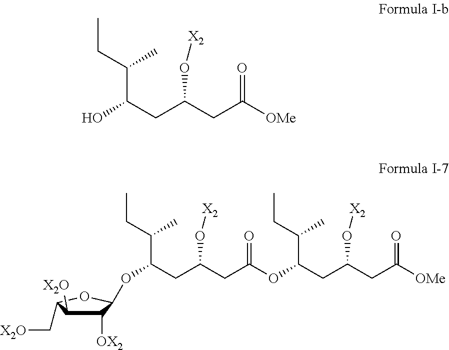
(j) performing hydrolysis of Formula I-7 to obtain Formula I.

5. The method of claim 4, wherein Formula I-a is prepared by installing the second alcohol protecting groups to L-arabinose, and then selectively removing one of the second alcohol protecting groups.

6. The method of claim 4, wherein Formula I-b is prepared by treating Formula I-3 with dimethyl carbonate and sodium methoxide, and then treating with Pd/C and hydrogen.

7. A method of preparing a compound of Formula IA, wherein apart from replacing (S)-Aux. by (R)-Aux., the remaining steps are the same as the method of preparing a compound of Formula I of claim 1.

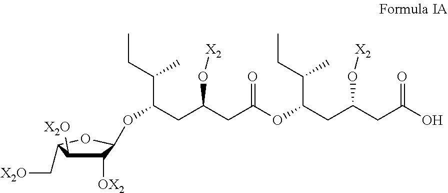

8. A method of preparing a compound of Formula IB, wherein apart from replacing hydroxylation by acetylation and without the inversion process, the remaining steps are the same as the method of preparing a compound of Formula I of claim 1.

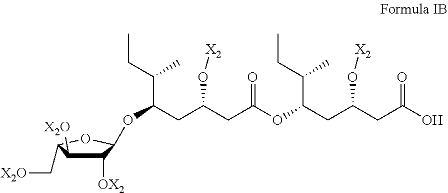

9. A method of preparing a compound of Formula IC, wherein apart from replacing hydroxylation by acetylation, replacing (S)-Aux. by (R)-Aux. and without the inversion process, the remaining steps are the same as the method of preparing a compound of Formula I of claim 1.

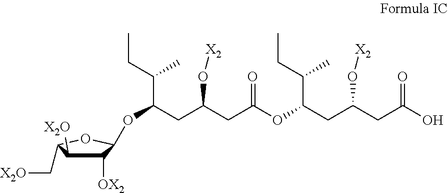

10. A method of preparing a compound of Formula ID, wherein apart from replacing L-isoleucine by D-isoleucine, replacing hydroxylation by acetylation and without the inversion process, the remaining steps are the same as the method of preparing a compound of Formula I of claim 1.

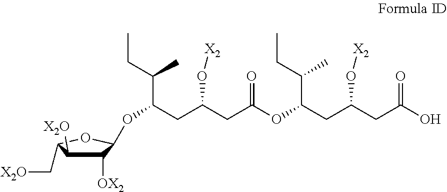

11. A method of preparing a compound of Formula IE, wherein apart from replacing L-isoleucine by D-isoleucine, replacing hydroxylation by acetylation, replacing (S)-Aux. by (R)-Aux. and without the inversion process, the remaining steps are the same as the method of preparing a compound of Formula I of claim 1.

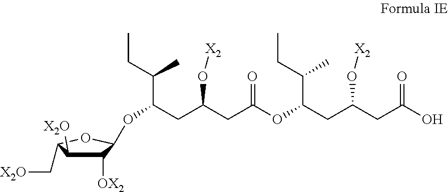

12. A method of preparing a compound of Formula IF, wherein apart from replacing L-isoleucine by D-isoleucine, the remaining steps are the same as the method of preparing a compound of Formula I of claim 1.

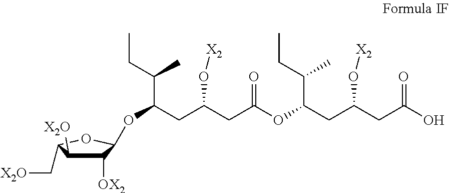

13. A method of preparing a compound of Formula IG, wherein apart from replacing L-isoleucine by D-isoleucine and replacing (S)-Aux by (R)-Aux., the remaining steps are the same as the method of preparing a compound of Formula I of claim 1. [chemical structure]
